SEQUENCE LISTING (76 sequences)

SEQ ID NO: 1 - DNA, 476 bp, Artificial Sequence
  Synthesized sequence
tttttgtagaaatgtcttggtgtcctcgaccaatcaggtagccatccctgaaatacctggctccgtggcaacaccgaacgacctgctggcaacgttaaattctccggggtaaaacttaaatgtggagtaatagaaccagaaacgtctcttcccttctctctccttccaccgcccgttaccgtccctaggaaattttactctgctggagagcttcttctacggcccccttgcagcaatgctcttcccagcattacgttgcgggtaaaacggaggtcgtgtacccgacctagcagcccagggatggaaagtcccggccgtcgctggcaataactgcgggcggacgcatgtcttgagattattggaaaccaccagaatcgaatataaaaggcgaacacctttcccaattttggtttctcctgacccaaagactttaaatttaatttatttgtccctatttcaatcaattgaacaactat

SEQ ID NO: 2 - DNA, 477 bp, Artificial Sequence
  Synthesized sequence
tttttgtagaaatgtcttggtgtcctcgtccaatcaggtagccatctctgaaatatctggctccgttgcaactccgaacgacctgctggcaacgtaaaattctccggggtaaaacttaaatgtggagtaatggaaccagaaacgtctcttcccttctctctccttccaccgcccgttaccgtccctaggaaattttactctgctggagagcttcttctacggcccccttgcagcaatgctcttcccagcattacgttgcgggtaaaacggaggtcgtgtacccgacctagcagcccagggatggaaaagtcccggccgtcgctggcaataatagcgggcggacgcatgtcatgagattattggaaaccaccagaatcgaatataaaaggcgaacacctttcccaattttggtttctcctgacccaaagactttaaatttaatttatttgtccctatttcaatcaattgaacaactat

SEQ ID NO: 3 - DNA, 375 bp, Artificial Sequence
  Synthesized sequence
atggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactga

SEQ ID NO: 4 - protein, 124 aa, Artificial Sequence
  Synthesized sequence
MAKLTSAVPVLTARDVAGAVEFWTDRLGFSRDFVEDDFAGVVRDDVTLFISAVQDQVVPDNTLAWVWVRGLDELYAEWSEVVSTNFRDASGPAMTEIGEQPWGREFALRDPAGNCVHFVAEEQD

SEQ ID NO: 5 - DNA, 861 bp, Artificial Sequence
  Synthesized sequence
atgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaa

SEQ ID NO: 6 - protein, 271 aa, Artificial Sequence
  Synthesized sequence
MSHIQRETSCSRPRLNSNMDADLYGYKWARDNVGQSGATIYRLYGKPDAPELFLKHGKGSVANDVTDEMVRLNWLTEFMPLPTIKHFIRTPDDAWLLTTAIPGKTAFQVLEEYPDSGENIVDALAVFLRRLHSIPVCNCPFNSDRVFRLAQAQSRMNNGLVDASDFDDERNGWPVEQVWKEMHKLLPFSPDSVVTHGDFSLDNLIFDEGKLIGCIDVGRVGIADRYQDLAILWNCLGEFSPSLQKRLFQKYGIDNPDMNKLQFHLMLDEFF

SEQ ID NO: 7 - DNA, 2079 bp, Arabidopsis thaliana
atgaccagcgccctgtatgcaagtgatctgtttaagcaactgaaaagcattatgggcaccgatagtctgagtgatgatgttgttctggttattgccaccaccagtctggccctggttgcaggttttgtggttctgctgtggaaaaagactaccgccgatcgcagtggtgaactgaaaccgctgatgattccgaaaagcctgatggccaaagatgaagatgatgatctggatctgggcagcggtaaaacccgtgttagcattttctttggcacccagaccggcaccgccgaaggctttgccaaagcactgagcgaagaaattaaggcccgctatgaaaaagcagcagttaaagtgattgatctggatgattatgccgccgatgatgatcagtatgaagaaaaactgaaaaaggaaaccctggcatttttctgtgtggcaacctatggcgatggtgaaccgaccgataatgcagcacgtttttataaatggtttaccgaagaaaacgagcgtgatattaagctgcaacagctggcctatggcgtttttgcactgggtaatcgtcagtatgaacattttaataagatcggcattgtgctggatgaagaactgtgtaaaaaaggcgcaaaacgtctgattgaagtgggcctgggcgatgatgatcaaagtattgaagatgatttcaacgcatggaaagaaagcctgtggagtgaactggataaactgctgaaagatgaagacgataaaagcgttgcaaccccgtataccgccgttattccggaatatcgcgtggtgacccatgatccgcgctttaccacccagaaaagtatggaaagtaatgttgccaatggtaataccaccattgatattcatcatccgtgtcgcgtggatgtggccgttcagaaagaactgcatacccatgaaagcgatcgtagctgtattcatctggaatttgatattagtcgtaccggtattacctatgaaaccggtgaccatgttggcgtttatgccgaaaatcatgttgaaattgtggaagaagcaggtaaactgctgggccatagtctggatctggtttttagtattcatgcagataaagaagacggtagtccgctggaaagcgccgttccgccgccgtttccgggtccttgcaccctgggtacaggcctggcacgctatgccgatctgctgaatccgccgcgtaaaagcgcactggttgccctggccgcctatgcaaccgaaccgagcgaagcagaaaaactgaagcatctgaccagcccggatggtaaagatgaatatagccagtggattgttgcaagccagcgtagcctgctggaagttatggccgcctttccgagtgcaaaaccgccgctgggtgttttctttgccgcaatcgctccgcgtctgcaaccgcgctattatagcattagcagtagtccgcgtctggcaccgagccgcgtgcatgttaccagtgcactggtttatggcccgaccccgaccggccgtattcataaaggtgtgtgtagcacctggatgaaaaatgcagttccggccgaaaaaagtcatgaatgtagtggcgccccgatttttattcgcgccagcaattttaaactgccgagtaatccgagcaccccgattgttatggtgggtccgggcaccggtctggcaccgtttcgcggttttctgcaagaacgcatggcactgaaagaagatggcgaagaactgggcagtagtctgctgtttttcggttgtcgtaatcgccagatggattttatctatgaagatgaactgaacaacttcgttgatcagggcgtgattagcgaactgattatggcctttagtcgtgaaggtgcccagaaagaatatgttcagcataaaatgatggagaaagccgcccaggtttgggatctgattaaggaagaaggctatctgtatgtgtgtggcgatgcaaaaggcatggcacgtgatgttcatcgtaccctgcataccattgttcaggaacaggaaggtgtgagcagcagcgaagcagaggccattgtgaaaaaactgcaaaccgaaggtcgttatctgcgtgatgtgtggtaa

SEQ ID NO: 8 - protein, 692 aa, Arabidopsis thaliana [sequence omitted]

SEQ ID NO: 9 - DNA, 1539 bp, Cosmos sulphureus
atgaccattctgccgctgctgctgtatccgagtctgaccgcactgctgctgtacgtgctgctgaatctgcgcccgcgccatccgaatcgtctgccgcctggtccgagcccgtggcctattgtgggtaatctgccgcatctgggcgcaagtccgcatcagagcctggcaaccctggcagcaaaatatggcccgctgatgtatctgcgtctgggttttgttgatgttgtggtggcagccagtgccagtgtggcagcacagtttctgaaagtgcatgatctgaattttgccagtcgcccgctgagcagcggtggcaaatatattgcctataattatcaggatatggtgtttgcaccgtatggtccgcgctggcgtatgctgcgcaaaatttgtagcgttcacatgtttagtgcaaaagcaatggatggctttcgccatgttcgtcaggaagaagttgccattctgacccgcaccctggttagtgccggtaaaagtccggttaaactgggccagattctgaatgtttgcaccaccaatgccctggcacgtgttgtgctgggtcgtcgtgtgtttgcagatggcagcgcaggtggtgacccgaaagcagatgagtttaaagatatggttgttgaactgatggtgctggcaggcgaatttcatattggtgactttattccggccctggattggctggatctgcagggtattaagaataagatgaaaaaactgcacgcccgttttgatagttttctgcatggcattctggaagaacataaaagcggtaaatttggcgcaccgagtcatggtgacctgctgagtaccctgattagtctgaaagatgatgcagatggtgaaggcggcaaactgagcgatgttgaaattaaggccctgctgctgaatctttttgtggccggcaccgataccagcagtagcaccgttgaatgggcaattgcagaactgattcgtcatccgaaactgctgaaacaggcacagaaagaaatggataatgtggtgggccgcgatcgtctggtgaccgaactggatctgaatgaactgaattttctgcaggcaattgttaaagaaacctttcgtctgcatccgagtaccccgctgagcctgccgcgcattgcaagcgaaagctgtgaagtggatggctattatattccgaaaggcagtaccctgctggtgaatgtgtgggccattgcacgtgatccgaatgtgtgggcagatccgctggaatttcgtccgatgcgttttctgccgggcggtgaaaaaccgaatgttgatgtgcagggtaataattttgaagttattccgtttggcgcaggccgtcgtatttgcgtgggtattagtctgggtctgcgcatggtgcagctgctggtggcaaccctggtgcagacctttgattgggaactggcaaatggtctgaatccggaaaaactgaatatggatgaagcatttggcctgaccctgcagaaagccgaaccgctgatggtgcatccgatgccgcgcctggcaccgcatgtttatggtagtcattaa

SEQ ID NO: 10 - protein, 512 aa, Cosmos sulphureus
MTILPLLLYPSLTALLLYVLLNLRPRHPNRLPPGPSPWPIVGNLPHLGASPHQSLATLAAKYGPLMYLRLGFVDVVVAASASVAAQFLKVHDLNFASRPLSSGGKYIAYNYQDMVFAPYGPRWRMLRKICSVHMFSAKAMDGFRHVRQEEVAILTRTLVSAGKSPVKLGQILNVCTTNALARVVLGRRVFADGSAGGDPKADEFKDMVVELMVLAGEFHIGDFIPALDWLDLQGIKNKMKKLHARFDSFLHGILEEHKSGKFGAPSHGDLLSTLISLKDDADGEGGKLSDVEIKALLLNLFVAGTDTSSSTVEWAIAELIRHPKLLKQAQKEMDNVVGRDRLVTELDLNELNFLQAIVKETFRLHPSTPLSLPRIASESCEVDGYYIPKGSTLLVNVWAIARDPNVWADPLEFRPMRFLPGGEKPNVDVQGNNFEVIPFGAGRRICVGISLGLRMVQLLVATLVQTFDWELANGLNPEKLNMDEAFGLTLQKAEPLMVHPMPRLAPHVYGSH

SEQ ID NO: 11 - DNA, 1155 bp, Saccharomyces cerevisiae
atgtccaagagcaaaactttcttatttacctctgaatccgtcggtgaaggtcacccagacaagatttgtgaccaagtttctgatgctattttggacgcttgtttagaacaagatccattctccaaggttgcctgtgaaacagctgccaaaactggtatgattatggttttcggtgaaattaccaccaaagctagacttgactaccaacaaatagtaagagataccatcaagaagattggttatgacgattctgccaagggtttcgactacaagacatgtaatgttttagtagctatcgaacaacaatctccagatatcgctcaaggtctgcactatgaaaagagcttagaagacttaggtgctggtgaccaaggtataatgtttggttacgctacagacgaaactccagaagggttaccattgaccattcttttggctcacaaattgaacatggctatggcagatgctagaagagatggttctctcccatggttgagaccagacacaaagactcaagtcactgtcgaatacgaagacgacaatggtagatgggttccaaagaggatagataccgttgttatttctgctcaacatgctgatgaaatttccaccgctgacttgagaactcaacttcaaaaagatattgttgaaaaggtcataccaaaggatatgttagacgaaaataccaaatatttcatccaaccatccggtagattcgtcatcggtggtcctcaaggtgacgctggtttgaccggtagaaagattattgtcgacgcttacggtggtgcctcatccgtcggtggtggtgccttctccggtaaggactattccaaggtcgatcgttccgctgcttacgctgctagatgggttgccaagtctctagttgccgctggtttgtgtaagagagtccaagtccaattttcatatgctattggtattgctgaaccattgtctttacatgtggacacctatggtacagctacaaaatcagatgacgaaatcattgaaattattaagaagaacttcgacttgagaccaggtgtgttagtaaaggaattagatttggctagaccaatttacttaccaaccgcttcttatggtcacttcactaatcaagagtactcatgggaaaaaccaaagaaattggaattttaa

SEQ ID NO: 12 - protein, 384 aa, Saccharomyces cerevisiae
MSKSKTFLFTSESVGEGHPDKICDQVSDAILDACLEQDPFSKVACETAAKTGMIMVFGEITTKARLDYQQIVRDTIKKIGYDDSAKGFDYKTCNVLVAIEQQSPDIAQGLHYEKSLEDLGAGDQGIMFGYATDETPEGLPLTILLAHKLNMAMADARRDGSLPWLRPDTKTQVTVEYEDDNGRWVPKRIDTVVISAQHADEISTADLRTQLQKDIVEKVIPKDMLDENTKYFIQPSGRFVIGGPQGDAGLTGRKIIVDAYGGASSVGGGAFSGKDYSKVDRSAAYAARWVAKSLVAAGLCKRVQVQFSYAIGIAEPLSLHVDTYGTATKSDDEIIEIIKKNFDLRPGVLVKELDLARPIYLPTASYGHFTNQEYSWEKPKKLEF

SEQ ID NO: 13 - DNA, 663 bp, Myxococcus xanthus
atgatccaccacgtcgaattgacacagtccgtcctgcagtacatccgcgacagttccgtgagggacaacgacatccttcgcgacctgcgagaggagacgtcgaagctccccctgcgcacgatgcagattcctcccgagcaggggcagctcctgagcctgctggtgcgcctcatcggcgcgcgcaagacgctcgaggtgggcgtcttcaccggctatagcaccctgtgtgccgcgctcgcgctgcccgccgatgggcgcgtcatcgcgtgcgacctgagcgaggagtgggtctccatcgcgcgccgctactggcagcgggcgggcgtggcggatcgcatcgaggtccggcttggcgacgcccaccattccctggaggcgctggtcggcagcgagcaccgcgggacgttcgacctcgcgttcatcgatgcggacaaggagagctacgacttctactacgagcacgcgctgcggctggtgcgtcccggcgggttgatcatcctcgacaacacgctgtggtccgggaaggtcgccgacccgagcgtcgtcggggacccggagacggactcgctgcggcgcatcaacgccaagctcctgactgacgaacgggtggacctcagcatgctcccaatcgctgacggactgaccctcgctcgcaagcgctag

SEQ ID NO: 14 - protein, 220 aa, Myxococcus xanthus
MIHHVELTQSVLQYIRDSSVRDNDILRDLREETSKLPLRTMQIPPEQGQLLSLLVRLIGARKTLEVGVFTGYSTLCAALALPADGRVIACDLSEEWVSIARRYWQRAGVADRIEVRLGDAHHSLEALVGSEHRGTFDLAFIDADKESYDFYYEHALRLVRPGGLIILDNTLWSGKVADPSVVGDPETDSLRRINAKLLTDERVDLSMLPIADGLTLARKR

SEQ ID NO: 15 - DNA, 1095 bp, Pinus sylvestris
atgggttctgcgtctgaatcttctgaaatgaacgcgaaaatcgttaacgaagacgaatggctgctgggtatggaactgggtaacttctcttgcgttccgatggcgatgaaagcggcgatcgaactggacgttctgcagatcatcgcgaacgcgggtaacggtgttcagctgtctccgcgtcagatcgttgcgcacatcccgaccaccaacccggacgcggcgatcaccctggaccgtatcctgcgtgttctggcgtctcactctgttctgtcttgctctgttaccaccgacgaaaacggtaaagcggaacgtctgtacggtctgaccccgctgtgcaaatacctggttaaaaaccaggacggtgtttctctggcgccgctggttctgatgaaccaggacaaagttctgatggaatcttggtactacctgaaagacgcggttctggacggttctcagccgttcaccaaagcgcacggtatgaacgcgttcgaatacccggcgatggaccagcgtttcaaccgtgttttcaaccgtggtatggcggaacactctaccatgctgatgaacaaaatcctggacacctacgaaggtttcaaagaagttcaggaactggttgacgttggtggtggtgttggttctaccctgaacctgatcgtttctaaatacccgcacatctctggtatcaacttcgacatgccgcacgttgttgcggacgcgccgcactacccggcggttaaacacgttggtggtgacatgttcgactctgttccgtctggtcaggcgatcttcatgaaatggattttacacgactggtctgacgaccactgcctgcgtctgctgaaaaactgccacaaagcgctgccggaaaaaggtaaagttatcgttgttgacaccatcctgccggttgcggcggaaacctctccgtacgcgcgtcagggtttccacatcgacctgctgatgctggcgtacaacccgggtggtaaagaacgtaccgaacaggaattccgtgacctggcgaaagaagttggtttcgcgggtggtgttaaaccggtttgctgcgttaacggtcactgggttatggaattccacaaatag

SEQ ID NO: 16 - protein, 364 aa, Pinus sylvestris
MGSASESSEMNAKIVNEDEWLLGMELGNFSCVPMAMKAAIELDVLQIIANAGNGVQLSPRQIVAHIPTTNPDAAITLDRILRVLASHSVLSCSVTTDENGKAERLYGLTPLCKYLVKNQDGVSLAPLVLMNQDKVLMESWYYLKDAVLDGSQPFTKAHGMNAFEYPAMDQRFNRVFNRGMAEHSTMLMNKILDTYEGFKEVQELVDVGGGVGSTLNLIVSKYPHISGINFDMPHVVADAPHYPAVKHVGGDMFDSVPSGQAIFMKWILHDWSDDHCLRLLKNCHKALPEKGKVIVVDTILPVAAETSPYARQGFHIDLLMLAYNPGGKERTEQEFRDLAKEVGFAGGVKPVCCVNGHWVMEFHK

SEQ ID NO: 17 - DNA, 22 bp, Artificial Sequence
  Synthesized sequence
cgaaacgatgagatttccttca

SEQ ID NO: 18 - DNA, 25 bp, Artificial Sequence
  Synthesized sequence
agatctgatctcatgaccaaaatcc

SEQ ID NO: 19 - DNA, 51 bp, Artificial Sequence
  Synthesized sequence
ggattttggtcatgagatcagatcttttttgtagaaatgtcttggtgtcct

SEQ ID NO: 20 - DNA, 55 bp, Artificial Sequence
  Synthesized sequence
aattgaaggaaatctcatcgtttcgatagttgttcaattgattgaaatagggaca

SEQ ID NO: 21 - DNA, 19 bp, Artificial Sequence
  Synthesized sequence
cacgtccgacggcggccca

SEQ ID NO: 22 - DNA, 22 bp, Artificial Sequence
  Synthesized sequence
ggtttagttcctcaccttgtcg

SEQ ID NO: 23 - DNA, 48 bp, Artificial Sequence
  Synthesized sequence
atacgacaaggtgaggaactaaaccatgagccatattcaacgggaaac

SEQ ID NO: 24 - DNA, 48 bp, Artificial Sequence
  Synthesized sequence
gacccgtgggccgccgtcggacgtgttagaaaaactcatcgagcatca

SEQ ID NO: 25 - DNA, 24 bp, Artificial Sequence
  Synthesized sequence
gtttgtagccttagacatgactgt

SEQ ID NO: 26 - DNA, 37 bp, Artificial Sequence
  Synthesized sequence
cgtttcgatagttgttcaattgattgaaatagggaca

SEQ ID NO: 27 - DNA, 47 bp, Artificial Sequence
  Synthesized sequence
aatcaattgaacaactatcgaaacgatgaccagcgccctgtatgcaa

SEQ ID NO: 28 - DNA, 48 bp, Artificial Sequence
  Synthesized sequence
aacagtcatgtctaaggctacaaacttaccacacatcacgcagataac

SEQ ID NO: 29 - DNA, 45 bp, Artificial Sequence
  Synthesized sequence
aatcaattgaacaactatcgaaacgatgaccattctgccgctgct

SEQ ID NO: 30 - DNA, 50 bp, Artificial Sequence
  Synthesized sequence
aacagtcatgtctaaggctacaaacttaatgactaccataaacatgcggt

SEQ ID NO: 31 - DNA, 48 bp, Artificial Sequence
  Synthesized sequence
caatcaattgaacaactatcgaaacgatgtccaagagcaaaactttct

SEQ ID NO: 32 - DNA, 56 bp, Artificial Sequence
  Synthesized sequence
gaacagtcatgtctaaggctacaaacttaaaattccaatttctttggtttttccca

SEQ ID NO: 33 - DNA, 47 bp, Artificial Sequence
  Synthesized sequence
aatcaattgaacaactatcgaaacgatgatccaccacgtcgaattga

SEQ ID NO: 34 - DNA, 47 bp, Artificial Sequence
  Synthesized sequence
aacagtcatgtctaaggctacaaacctagcgcttgcgagcgagggtc

SEQ ID NO: 35 - DNA, 47 bp, Artificial Sequence
  Synthesized sequence
aatcaattgaacaactatcgaaacgatgggttctgcgtctgaatctt

SEQ ID NO: 36 - DNA, 49 bp, Artificial Sequence
  Synthesized sequence
aacagtcatgtctaaggctacaaacctatttgtggaattccataaccca

SEQ ID NO: 37 - DNA, 1203 bp, Bacillus subtilis
atgagcaagaatcgtcgtctgtttacctcagaatcggtgactgaaggccatccggataaaatctgcgatcagattagcgattccattctggacgagattctgaagaaagacccaaatgcgcgcgttgcatgcgaaacgtctgtcaccaccgggttggtcttagtgagcggcgaaattacaacttccacgtatgtggacattccgaaaaccgttcggcagaccatcaaagagattggctacactcgcgcgaaatacggatttgacgcggaaacctgtgcagttctgacctcgattgacgaacagtcagcggatattgcaatgggtgtcgaccaagcccttgaagcccgcgaaggaacgatgtcagacgaagaaattgaagccattggggctggcgatcaaggcctgatgttcggttatgcgtgcaacgaaaccaaagagttaatgccgctgcccatcagtctggcgcacaaactggcacgtcgcttgagcgaagtgcgcaaagaggatatcctgccgtatttacgccctgatgggaaaacccaggtcaccgtggaatacgatgagaacaacaaaccggtacgcattgacgcgattgtgatttctacgcagcatcatccagagatcactcttgaacagatccaacgcaacatcaaagaacacgtgatcaatccggtagttccggaagaactgatcgacgaagaaaccaaatacttcatcaacccgacagggcgttttgtaattggcggtccacagggtgatgctggccttacaggccgcaaaatcattgtggatacctatggtgggtatgcgcgccatggaggtggtgcctttagtggcaaggatgcaacgaaagtggatcggagcgctgcatatgccgcgcgttatgttgcgaagaacattgttgctgcggagttagcagattcgtgtgaagtccagttggcctatgccattggtgttgcccaacctgtcagcatttccatcaacacattcggttctggcaaagcgagtgaggagaaactgatcgaagtggtgcgcaataatttcgatctccgtcctgccggcattattaagatgctcgatctccgccgtccgatctacaaacaaacggctgcctacggtcattttggccgtcacgacgtagatctgccctgggaacgtacggataaggcggaacagctgcgcaaagaggctctgggcgagtaa

SEQ ID NO: 38 - protein, 400 aa, Bacillus subtilis
MSKNRRLFTSESVTEGHPDKICDQISDSILDEILKKDPNARVACETSVTTGLVLVSGEITTSTYVDIPKTVRQTIKEIGYTRAKYGFDAETCAVLTSIDEQSADIAMGVDQALEAREGTMSDEEIEAIGAGDQGLMFGYACNETKELMPLPISLAHKLARRLSEVRKEDILPYLRPDGKTQVTVEYDENNKPVRIDAIVISTQHHPEITLEQIQRNIKEHVINPVVPEELIDEETKYFINPTGRFVIGGPQGDAGLTGRKIIVDTYGGYARHGGGAFSGKDATKVDRSAAYAARYVAKNIVAAELADSCEVQLAYAIGVAQPVSISINTFGSGKASEEKLIEVVRNNFDLRPAGIIKMLDLRRPIYKQTAAYGHFGRHDVDLPWERTDKAEQLRKEALGE

SEQ ID NO: 39 - DNA, 1203 bp, Bacillus subtilis
atgagcaagaatcgtcgtctgtttacctcagaatcggtgactgaaggccatccggataaaatctgcgatcagattagcgattccattctggacgagattctgaagaaagacccaaatgcgcgcgttgcatgcgaaacgtctgtcaccaccgggttggtcttagtgagcggcgaaattacaacttccacgtatgtggacattccgaaaaccgttcggcagaccatcaaagagattggctacactcgcgcgaaatacggatttgacgcggaaacctgtgcagttctgacctcgattgacgaacagtcagcggatattgcaatgggtgtcgaccaagcccttgaagcccgcgaaggaacgatgtcagacgaagaaattgaagccattggggctggcgatcaaggcctgatgttcggttatgcgtgcaacgaaaccaaagagttaatgccgctgcccatcagtctggcgcacaaactggcacgtcgcttgagcgaagtgcgcaaagaggatatcctgccgtatttacgccctgatgggaaaacccaggtcaccgtggaatacgatgagaacaacaaaccggtacgcattgacgcgattgtgatttctacgcagcatcatccagagatcactcttgaacagatccaacgcaacatcaaagaacacgtgatcaatccggtagttccggaagaactgatcgacgaagaaaccaaatacttcatcaacccgacagggcgttttgtaattggcggtccacagggtgatgctggccttacaggccgcaaaatcattgtggatacctatggtgggtatgcgcgccatggaggtggtgcctttagtggcaaggatgcaacgaaagtggatcggagcgctgcatatgccgcgcgttatgttgcgaagaacattgttgctgcggagttagcagattcgtgtgaagtccagttggcctatgccgttggtgttgcccaacctgtcagcatttccatcaacacattcggttctggcaaagcgagtgaggagaaactgatcgaagtggtgcgcaataatttcgatctccgtcctgccggcattattaagatgctcgatctccgccgtccgatctacaaacaaacggctgcctacggtcattttggccgtcacgacgtagatctgccctgggaacgtacggataaggcggaacagctgcgcaaagaggctctgggcgagtaa

SEQ ID NO: 40 - protein, 400 aa, Bacillus subtilis
MSKNRRLFTSESVTEGHPDKICDQISDSILDEILKKDPNARVACETSVTTGLVLVSGEITTSTYVDIPKTVRQTIKEIGYTRAKYGFDAETCAVLTSIDEQSADIAMGVDQALEAREGTMSDEEIEAIGAGDQGLMFGYACNETKELMPLPISLAHKLARRLSEVRKEDILPYLRPDGKTQVTVEYDENNKPVRIDAIVISTQHHPEITLEQIQRNIKEHVINPVVPEELIDEETKYFINPTGRFVIGGPQGDAGLTGRKIIVDTYGGYARHGGGAFSGKDATKVDRSAAYAARYVAKNVVAAELADSCEVQLAYAIGVAQPVSISINTFGSGKASEEKLIEVVRNNFDLRPAGIIKMLDLRRPIYKQTAAYGHFGRHDVDLPWERTDKAEQLRKEALGE

SEQ ID NO: 41 - DNA, 1155 bp, Escherichia coli
atggcaaaacacctttttacgtccgagtccgtctctgaagggcatcctgacaaaattgctgaccaaatttctgatgccgttttagacgcgatcctcgaacaggatccgaaagcacgcgttgcttgcgaaacctacgtaaaaaccggcatggttttagttggcggcgaaatcaccaccagcgcctgggtagacatcgaagagatcacccgtaacaccgttcgcgaaattggctatgtgcattccgacatgggctttgacgctaactcctgtgcggttctgagcgctatcggcaaacagtctcctgacatcaaccagggcgttgaccgtgccgatccgctggaacagggcgcgggtgaccagggtctgatgtttggctacgcaactaatgaaaccgacgtgctgatgccagcacctatcacctatgcacaccgtctggtacagcgtcaggctgaagtgcgtaaaaacggcactctgccgtggctgcgcccggacgcgaaaagccaggtgacttttcagtatgacgacggcaaaatcgttggtatcgatgctgtcgtgctttccactcagcactctgaagagatcgaccagaaatcgctgcaagaagcggtaatggaagagatcatcaagccaattctgcccgctgaatggctgacttctgccaccaaattcttcatcaacccgaccggtcgtttcgttatcggtggcccaatgggtgactgcggtctgactggtcgtaaaattatcgttgatacctacggcggcatggcgcgtcacggtggcggtgcattctctggtaaagatccatcaaaagtggaccgttccgcagcctacgcagcacgttatgtcgcgaaaaacatcgttgctgctggcctggccgatcgttgtgaaattcaggtttcctacgcaatcggcgtggctgaaccgacctccatcatggtagaaactttcggtactgagaaagtgccttctgaacaactgaccctgctggtacgtgagttcttcgacctgcgcccatacggtctgattcagatgctggatctgctgcacccgatctacaaagaaaccgcagcatacggtcactttggtcgtgaacatttcccgtgggaaaaaaccgacaaagcgcagctgctgcgcgatgctgccggtctgaagtaa

SEQ ID NO: 42 - protein, 384 aa, Escherichia coli
MAKHLFTSESVSEGHPDKIADQISDAVLDAILEQDPKARVACETYVKTGMVLVGGEITTSAWVDIEEITRNTVREIGYVHSDMGFDANSCAVLSAIGKQSPDINQGVDRADPLEQGAGDQGLMFGYATNETDVLMPAPITYAHRLVQRQAEVRKNGTLPWLRPDAKSQVTFQYDDGKIVGIDAVVLSTQHSEEIDQKSLQEAVMEEIIKPILPAEWLTSATKFFINPTGRFVIGGPMGDCGLTGRKIIVDTYGGMARHGGGAFSGKDPSKVDRSAAYAARYVAKNIVAAGLADRCEIQVSYAIGVAEPTSIMVETFGTEKVPSEQLTLLVREFFDLRPYGLIQMLDLLHPIYKETAAYGHFGREHFPWEKTDKAQLLRDAAGLK

SEQ ID NO: 43 - DNA, 1236 bp, Streptomyces spectabilis
atgtcccgccgtctcttcacctcggagtccgtgaccgagggtcaccccgacaagatcgctgaccagatcagcgacaccattctcgacgcgcttctgcgtgaggacccgacgtcccgggtcgccgtcgaaacgctcatcaccaccggcctcgtgcacgtcgccggtgaggtcacgaccaaggcgtacgcgccgatcgcgcagctggtgcgcgagaagatcctcgagatcggatacgactcctcgaagaagggcttcgacggcgcctcctgcggcgtctcggtgtccatcggcgcgcagtccccggacatcgcgcagggcgtcgacacggcgtacgagagccgtgtcgagggcgacgaggacgagctcgaccggcagggcgccggtgaccagggcctgatgttcggctacgcctgcgacgagaccccggagctgatgccgctcccgatccacctcgcgcaccgcctctcgcgccgcctctccgaggtgcgcaagaacgggaccatcccctacctgcgccccgacggcaagacccaggtcaccatcgagtacgacggcgacaaggccgtccgcctcgacacggtcgtcgtctcctcgcagcacgcgtcggacatcgacctggagtcgctgctcgcccccgacatccgcgagttcgtcgtcgagccggagctcaaggccctggtcgaggacggcatcaagctggagaccgagggctaccgcctcctggtcaacccgaccggccgcttcgagatcggcggcccgatgggtgacgcgggcctgaccggccgcaagatcatcatcgacacctacggcggcatgtcccgccacggcggcggcgccttctccggcaaggacccgtccaaggtggaccgctcggcggcgtacgcgatgcgctgggtcgccaagaacgtggtggccgcgggcctcgcctcgcgctgcgaggtccaggtcgcctacgccatcggcaaggccgagccggtcggtctgttcgtcgagaccttcggcaccaacacgatcgacacggacaagatcgagcaggccatcagcgaggtcttcgacctccgcccggccgcgatcatccgcgacctcgacctgctccgcccgatctactcccagaccgccgcgtacggccacttcggccgctcgctgccggagttcacctgggagaagacggaccgcgtggacggctgcggaaggccgccggtctggagagctgatctcctgccgcttgttcactga

SEQ ID NO: 44 - protein, 411 aa, Streptomyces spectabilis
MSRRLFTSESVTEGHPDKIADQISDTILDALLREDPTSRVAVETLITTGLVHVAGEVTTKAYAPIAQLVREKILEIGYDSSKKGFDGASCGVSVSIGAQSPDIAQGVDTAYESRVEGDEDELDRQGAGDQGLMFGYACDETPELMPLPIHLAHRLSRRLSEVRKNGTIPYLRPDGKTQVTIEYDGDKAVRLDTVVVSSQHASDIDLESLLAPDIREFVVEPELKALVEDGIKLETEGYRLLVNPTGRFEIGGPMGDAGLTGRKIIIDTYGGMSRHGGGAFSGKDPSKVDRSAAYAMRWVAKNVVAAGLASRCEVQVAYAIGKAEPVGLFVETFGTNTIDTDKIEQAISEVFDLRPAAIIRDLDLLRPIYSQTAAYGHFGRSLPEFTWEKTDRVDGCGRPPVWRADLLPLVH

SEQ ID NO: 45 - DNA, 1518 bp, Saccharomyces cerevisiae
atgagtgaaggccccgtcaaattcgaaaaaaataccgtcatatctgtctttggtgcgtcaggtgatctggcaaagaagaagacttttcccgccttatttgggcttttcagagaaggttaccttgatccatctaccaagatcttcggttatgcccggtccaaattgtccatggaggaggacctgaagtcccgtgtcctaccccacttgaaaaaacctcacggtgaagccgatgactctaaggtcgaacagttcttcaagatggtcagctacatttcgggaaattacgacacagatgaaggcttcgacgaattaagaacgcagatcgagaaattcgagaaaagtgccaacgtcgatgtcccacaccgtctcttctatctggccttgccgccaagcgtttttttgacggtggccaagcagatcaagagtcgtgtgtacgcagagaatggcatcacccgtgtaatcgtagagaaacctttcggccacgacctggcctctgccagggagctgcaaaaaaacctggggcccctctttaaagaagaagagttgtacagaattgaccattacttgggtaaagagttggtcaagaatcttttagtcttgaggttcggtaaccagtttttgaatgcctcgtggaatagagacaacattcaaagcgttcagatttcgtttaaagagaggttcggcaccgaaggccgtggcggctatttcgactctataggcataatcagagacgtgatgcagaaccatctgttacaaatcatgactctcttgactatggaaagaccggtgtcttttgacccggaatctattcgtgacgaaaaggttaaggttctaaaggccgtggcccccatcgacacggacgacgtcctcttgggccagtacggtaaatctgaggacgggtctaagcccgcctacgtggatgatgacactgtagacaaggactctaaatgtgtcacttttgcagcaatgactttcaacatcgaaaacgagcgttgggagggcgtccccatcatgatgcgtgccggtaaggctttgaatgagtccaaggtggagatcagactgcagtacaaagcggtcgcatcgggtgtcttcaaagacattccaaataacgaactggtcatcagagtgcagcccgatgccgctgtgtacctaaagtttaatgctaagacccctggtctgtcaaatgctacccaagtcacagatctgaatctaacttacgcaagcaggtaccaagacttttggattccagaggcttacgaggtgttgataagagacgccctactgggtgaccattccaactttgtcagagatgacgaattggatatcagttggggcatattcaccccattactgaagcacatagagcgtccggacggtccaacaccggaaatttacccctacggatcaagaggtccaaagggattgaaggaatatatgcaaaaacacaagtatgttatgcccgaaaagcacccttacgcttggcccgtgactaagccagaagatacgaaggataattag

SEQ ID NO: 46 - protein, 505 aa, Saccharomyces cerevisiae
MSEGPVKFEKNTVISVFGASGDLAKKKTFPALFGLFREGYLDPSTKIFGYARSKLSMEEDLKSRVLPHLKKPHGEADDSKVEQFFKMVSYISGNYDTDEGFDELRTQIEKFEKSANVDVPHRLFYLALPPSVFLTVAKQIKSRVYAENGITRVIVEKPFGHDLASARELQKNLGPLFKEEELYRIDHYLGKELVKNLLVLRFGNQFLNASWNRDNIQSVQISFKERFGTEGRGGYFDSIGIIRDVMQNHLLQIMTLLTMERPVSFDPESIRDEKVKVLKAVAPIDTDDVLLGQYGKSEDGSKPAYVDDDTVDKDSKCVTFAAMTFNIENERWEGVPIMMRAGKALNESKVEIRLQYKAVASGVFKDIPNNELVIRVQPDAAVYLKFNAKTPGLSNATQVTDLNLTYASRYQDFWIPEAYEVLIRDALLGDHSNFVRDDELDISWGIFTPLLKHIERPDGPTPEIYPYGSRGPKGLKEYMQKHKYVMPEKHPYAWPVTKPEDTKDN

SEQ ID NO: 47 - DNA, 1515 bp, Komagataella phaffii
atgaccgatacgaaagccgtagaatttgtgggccacacagccattgtagtctttggagcttcaggggacctggctaagaagaagactttccctgccctcttcggactttaccgtgagggatacctgtccaacaaggtgaagattattggctatgctagatcaaagctggatgacaaggagttcaaggatagaattgtgggctatttcaagacaaagaacaagggcgacgaggacaaagttcaagaattcttaaagttgtgctcatatatttcagctccttatgacaaaccagatgggtatgaaaagttgaatgaaactattaacgaattcgaaaaggaaaacaacgtcgaacagtctcacaggttgttctacttagctttgcccccttctgttttcatacctgttgctacggaggtcaagaagtatgttcatccaggttctaaagggattgctcggattatcgtggaaaaacctttcgggcacgacttgcagtcagcagaagagcttttgaatgctttgaagccgatctggaaagaagaggaattgtttagaatcgaccactatctaggtaaggagatggttaagaatttgttggccttccgttttggaaacgcattcatcaatgcttcttgggacaacagacatatcagctgtatccaaatctcgttcaaggagccttttggaacagaaggtcgtggtggctattttgactcaattggtataataagagacgtcattcagaaccacttgcttcaagtgttaaccctcttaaccatggagagacccgtctctaatgaccctgaggctgttagagatgaaaaggttcgcattctgaagtcaatttctgagctagatttgaacgacgttttggtgggtcaatacggcaaatctgaggatggaaagaagccagcttatgtggatgatgaaactgttaagccaggttctaaatgtgtcacatttgcagccattggcttgcacatcaacacagaaaggtgggaaggtgtcccaatcattttaagagctggtaaggctttgaacgaaggtaaagttgagattagagtgcaatacaaacagtctactggatttctcaatgatattcagcgaaatgaattggtcatccgtgtgcagcctaacgaagccatgtacatgaaactgaactccaaagtcccaggtgtttcccaaaagactactgtcactgagctagacctcacttacaaagaccgttacgaaaacttttacattccagaggcatatgaatcacttatcagagatgctatgaagggagatcactctaattttgtcagagatgacgagttgatacaaagttggaagattttcactcctttactgtatcacttggagggccctgatgcaccggctccagaaatctatccctacggatccagaggtccagcttcattgaccaaattcttgcaagatcatgattacttctttgaatcacgcgacaattaccaatggccagtgacaagacccgatgtgctgcacaagatgtaa

SEQ ID NO: 48 - protein, 504 aa, Komagataella phaffii
MTDTKAVEFVGHTAIVVFGASGDLAKKKTFPALFGLYREGYLSNKVKIIGYARSKLDDKEFKDRIVGYFKTKNKGDEDKVQEFLKLCSYISAPYDKPDGYEKLNETINEFEKENNVEQSHRLFYLALPPSVFIPVATEVKKYVHPGSKGIARIIVEKPFGHDLQSAEELLNALKPIWKEEELFRIDHYLGKEMVKNLLAFRFGNAFINASWDNRHISCIQISFKEPFGTEGRGGYFDSIGIIRDVIQNHLLQVLTLLTMERPVSNDPEAVRDEKVRILKSISELDLNDVLVGQYGKSEDGKKPAYVDDETVKPGSKCVTFAAIGLHINTERWEGVPIILRAGKALNEGKVEIRVQYKQSTGFLNDIQRNELVIRVQPNEAMYMKLNSKVPGVSQKTTVTELDLTYKDRYENFYIPEAYESLIRDAMKGDHSNFVRDDELIQSWKIFTPLLYHLEGPDAPAPEIYPYGSRGPASLTKFLQDHDYFFESRDNYQWPVTRPDVLHKM

SEQ ID NO: 49 - DNA, 47 bp, Artificial Sequence
  Synthesized sequence
aatcaattgaacaactatcgaaacgatgagtgaaggccccgtcaaat

SEQ ID NO: 50 - DNA, 50 bp, Artificial Sequence
  Synthesized sequence
aacagtcatgtctaaggctacaaacctaattatccttcgtatcttctggc

SEQ ID NO: 51 - DNA, 1029 bp, Artificial Sequence
  Synthesized sequence
atgggtaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcgactccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaataa

SEQ ID NO: 52 - protein, 342 aa, Artificial Sequence
  Synthesized sequence
MGKKPELTATSVEKFLIEKFDSVSDLMQLSEGEESRAFSFDVGGRGYVLRVNSCADGFYKDRYVYRHFASAALPIPEVLDIGEFSESLTYCISRRAQGVTLQDLPETELPAVLQPVAEAMDAIAAADLSQTSGFGPFGPQGIGQYTTWRDFICAIADPHVYHWQTVMDDTVSASVAQALDELMLWAEDCPEVRHLVHADFGSNNVLTDNGRITAVIDWSEAMFGDSQYEVANIFFWRPWLACMEQQTRYFERRHPELAGSPRLRAYMLRIGLDQLYQSLVDGNFDDAAWAQGRCDAIVRSGAGTVGRTQIARRSAAVWTDGCVEVLADSGNRRPSTRPRAKE

SEQ ID NO: 53 - DNA, 48 bp, Artificial Sequence
  Synthesized sequence
atacgacaaggtgaggaactaaaccatgggtaaaaagcctgaactcac

SEQ ID NO: 54 - DNA, 46 bp, Artificial Sequence
  Synthesized sequence
gacccgtgggccgccgtcggacgtgttattcctttgccctcggacg

SEQ ID NO: 55 - DNA, 761 bp, Artificial Sequence
  MxSafC with tags
gcagcagccatcatcatcatcatcacagcagcggcctggtgccgcgcggcagccatatggctagcatgactggtggacagcaaatgggtcgcggatccatgatccaccacgtcgaattgacacagtccgtcctgcagtacatccgcgacagttccgtgagggacaacgacatccttcgcgacctgcgagaggagacgtcgaagctccccctgcgcacgatgcagattcctcccgagcaggggcagctcctgagcctgctggtgcgcctcatcggcgcgcgcaagacgctcgaggtgggcgtcttcaccggctatagcaccctgtgtgccgcgctcgcgctgcccgccgatgggcgcgtcatcgcgtgcgacctgagcgaggagtgggtctccatcgcgcgccgctactggcagcgggcgggcgtggcggatcgcatcgaggtccggcttggcgacgcccaccattccctggaggcgctggtcggcagcgagcaccgcgggacgttcgacctcgcgttcatcgatgcggacaaggagagctacgacttctactacgagcacgcgctgcggctggtgcgtcccggcgggttgatcatcctcgacaacacgctgtggtccgggaaggtcgccgacccgagcgtcgtcggggacccggagacggactcgctgcggcgcatcaacgccaagctcctgactgacgaacgggtggacctcagcatgctcccaatcgctgacggactgaccctcgctcgcaagcgctag

SEQ ID NO: 56 - DNA, 761 bp, Artificial Sequence
  MxSafC_L92Q
gcagcagccatcatcatcatcatcacagcagcggcctggtgccgcgcggcagccatatggctagcatgactggtggacagcaaatgggtcgcggatccatgatccaccacgtcgaattgacacagtccgtcctgcagtacatccgcgacagttccgtgagggacaacgacatccttcgcgacctgcgagaggagacgtcgaagctccccctgcgcacgatgcagattcctcccgagcaggggcagctcctgagcctgctggtgcgcctcatcggcgcgcgcaagacgctcgaggtgggcgtcttcaccggctatagcaccctgtgtgccgcgctcgcgctgcccgccgatgggcgcgtcatcgcgtgcgaccagagcgaggagtgggtctccatcgcgcgccgctactggcagcgggcgggcgtggcggatcgcatcgaggtccggcttggcgacgcccaccattccctggaggcgctggtcggcagcgagcaccgcgggacgttcgacctcgcgttcatcgatgcggacaaggagagctacgacttctactacgagcacgcgctgcggctggtgcgtcccggcgggttgatcatcctcgacaacacgctgtggtccgggaaggtcgccgacccgagcgtcgtcggggacccggagacggactcgctgcggcgcatcaacgccaagctcctgactgacgaacgggtggacctcagcatgctcccaatcgctgacggactgaccctcgctcgcaagcgctag

SEQ ID NO: 57 - DNA, 761 bp, Artificial Sequence
  MxSafC_W96A
gcagcagccatcatcatcatcatcacagcagcggcctggtgccgcgcggcagccatatggctagcatgactggtggacagcaaatgggtcgcggatccatgatccaccacgtcgaattgacacagtccgtcctgcagtacatccgcgacagttccgtgagggacaacgacatccttcgcgacctgcgagaggagacgtcgaagctccccctgcgcacgatgcagattcctcccgagcaggggcagctcctgagcctgctggtgcgcctcatcggcgcgcgcaagacgctcgaggtgggcgtcttcaccggctatagcaccctgtgtgccgcgctcgcgctgcccgccgatgggcgcgtcatcgcgtgcgacctgagcgaggaggcggtctccatcgcgcgccgctactggcagcgggcgggcgtggcggatcgcatcgaggtccggcttggcgacgcccaccattccctggaggcgctggtcggcagcgagcaccgcgggacgttcgacctcgcgttcatcgatgcggacaaggagagctacgacttctactacgagcacgcgctgcggctggtgcgtcccggcgggttgatcatcctcgacaacacgctgtggtccgggaaggtcgccgacccgagcgtcgtcggggacccggagacggactcgctgcggcgcatcaacgccaagctcctgactgacgaacgggtggacctcagcatgctcccaatcgctgacggactgaccctcgctcgcaagcgctag

SEQ ID NO: 58 - DNA, 761 bp, Artificial Sequence
  MxSafC_D119P
gcagcagccatcatcatcatcatcacagcagcggcctggtgccgcgcggcagccatatggctagcatgactggtggacagcaaatgggtcgcggatccatgatccaccacgtcgaattgacacagtccgtcctgcagtacatccgcgacagttccgtgagggacaacgacatccttcgcgacctgcgagaggagacgtcgaagctccccctgcgcacgatgcagattcctcccgagcaggggcagctcctgagcctgctggtgcgcctcatcggcgcgcgcaagacgctcgaggtgggcgtcttcaccggctatagcaccctgtgtgccgcgctcgcgctgcccgccgatgggcgcgtcatcgcgtgcgacctgagcgaggagtgggtctccatcgcgcgccgctactggcagcgggcgggcgtggcggatcgcatcgaggtccggcttggccccgcccaccattccctggaggcgctggtcggcagcgagcaccgcgggacgttcgacctcgcgttcatcgatgcggacaaggagagctacgacttctactacgagcacgcgctgcggctggtgcgtcccggcgggttgatcatcctcgacaacacgctgtggtccgggaaggtcgccgacccgagcgtcgtcggggacccggagacggactcgctgcggcgcatcaacgccaagctcctgactgacgaacgggtggacctcagcatgctcccaatcgctgacggactgaccctcgctcgcaagcgctag

SEQ ID NO: 59 - DNA, 761 bp, Artificial Sequence
  MxSafC_T40P
gcagcagccatcatcatcatcatcacagcagcggcctggtgccgcgcggcagccatatggctagcatgactggtggacagcaaatgggtcgcggatccatgatccaccacgtcgaattgacacagtccgtcctgcagtacatccgcgacagttccgtgagggacaacgacatccttcgcgacctgcgagaggagacgtcgaagctccccctgcgcccgatgcagattcctcccgagcaggggcagctcctgagcctgctggtgcgcctcatcggcgcgcgcaagacgctcgaggtgggcgtcttcaccggctatagcaccctgtgtgccgcgctcgcgctgcccgccgatgggcgcgtcatcgcgtgcgacctgagcgaggagtgggtctccatcgcgcgccgctactggcagcgggcgggcgtggcggatcgcatcgaggtccggcttggcgacgcccaccattccctggaggcgctggtcggcagcgagcaccgcgggacgttcgacctcgcgttcatcgatgcggacaaggagagctacgacttctactacgagcacgcgctgcggctggtgcgtcccggcgggttgatcatcctcgacaacacgctgtggtccgggaaggtcgccgacccgagcgtcgtcggggacccggagacggactcgctgcggcgcatcaacgccaagctcctgactgacgaacgggtggacctcagcatgctcccaatcgctgacggactgaccctcgctcgcaagcgctag

SEQ ID NO: 60 - DNA, 761 bp, Artificial Sequence
  MxSafC_S173H
gcagcagccatcatcatcatcatcacagcagcggcctggtgccgcgcggcagccatatggctagcatgactggtggacagcaaatgggtcgcggatccatgatccaccacgtcgaattgacacagtccgtcctgcagtacatccgcgacagttccgtgagggacaacgacatccttcgcgacctgcgagaggagacgtcgaagctccccctgcgcacgatgcagattcctcccgagcaggggcagctcctgagcctgctggtgcgcctcatcggcgcgcgcaagacgctcgaggtgggcgtcttcaccggctatagcaccctgtgtgccgcgctcgcgctgcccgccgatgggcgcgtcatcgcgtgcgacctgagcgaggagtgggtctccatcgcgcgccgctactggcagcgggcgggcgtggcggatcgcatcgaggtccggcttggcgacgcccaccattccctggaggcgctggtcggcagcgagcaccgcgggacgttcgacctcgcgttcatcgatgcggacaaggagagctacgacttctactacgagcacgcgctgcggctggtgcgtcccggcgggttgatcatcctcgacaacacgctgtggcacgggaaggtcgccgacccgagcgtcgtcggggacccggagacggactcgctgcggcgcatcaacgccaagctcctgactgacgaacgggtggacctcagcatgctcccaatcgctgacggactgaccctcgctcgcaagcgctag

SEQ ID NO: 61 - DNA, 761 bp, Artificial Sequence
  MxSafC_T40P_S173H
gcagcagccatcatcatcatcatcacagcagcggcctggtgccgcgcggcagccatatggctagcatgactggtggacagcaaatgggtcgcggatccatgatccaccacgtcgaattgacacagtccgtcctgcagtacatccgcgacagttccgtgagggacaacgacatccttcgcgacctgcgagaggagacgtcgaagctccccctgcgcccgatgcagattcctcccgagcaggggcagctcctgagcctgctggtgcgcctcatcggcgcgcgcaagacgctcgaggtgggcgtcttcaccggctatagcaccctgtgtgccgcgctcgcgctgcccgccgatgggcgcgtcatcgcgtgcgacctgagcgaggagtgggtctccatcgcgcgccgctactggcagcgggcgggcgtggcggatcgcatcgaggtccggcttggcgacgcccaccattccctggaggcgctggtcggcagcgagcaccgcgggacgttcgacctcgcgttcatcgatgcggacaaggagagctacgacttctactacgagcacgcgctgcggctggtgcgtcccggcgggttgatcatcctcgacaacacgctgtggcacgggaaggtcgccgacccgagcgtcgtcggggacccggagacggactcgctgcggcgcatcaacgccaagctcctgactgacgaacgggtggacctcagcatgctcccaatcgctgacggactgaccctcgctcgcaagcgctag

SEQ ID NO: 62 - DNA, 761 bp, Artificial Sequence
  MxSafC_M5
gcagcagccatcatcatcatcatcacagcagcggcctggtgccgcgcggcagccatatggctagcatgactggtggacagcaaatgggtcgcggatccatgatccaccacgtcgaattgacacagtccgtcctgcagtacatccgcgacagttccgtgagggacaacgacatccttcgcgacctgcgagaggagacgtcgaagctccccctgcgcccgatgcagattcctcccgagcaggggcagctcctgagcctgctggtgcgcctcatcggcgcgcgcaagacgctcgaggtgggcgtcttcaccggctatagcaccctgtgtgccgcgctcgcgctgcccgccgatgggcgcgtcatcgcgtgcgaccagagcgaggaggcggtctccatcgcgcgccgctactggcagcgggcgggcgtggcggatcgcatcgaggtccggcttggccccgcccaccattccctggaggcgctggtcggcagcgagcaccgcgggacgttcgacctcgcgttcatcgatgcggacaaggagagctacgacttctactacgagcacgcgctgcggctggtgcgtcccggcgggttgatcatcctcgacaacacgctgtggcacgggaaggtcgccgacccgagcgtcgtcggggacccggagacggactcgctgcggcgcatcaacgccaagctcctgactgacgaacgggtggacctcagcatgctcccaatcgctgacggactgaccctcgctcgcaagcgctag

SEQ ID NO: 63 - protein, 252 aa, Artificial Sequence
  MxSafC_L92Q with tags
SSHHHHHHSSGLVPRGSHMASMTGGQQMGRGSMIHHVELTQSVLQYIRDSSVRDNDILRDLREETSKLPLRTMQIPPEQGQLLSLLVRLIGARKTLEVGVFTGYSTLCAALALPADGRVIACDQSEEWVSIARRYWQRAGVADRIEVRLGDAHHSLEALVGSEHRGTFDLAFIDADKESYDFYYEHALRLVRPGGLIILDNTLWSGKVADPSVVGDPETDSLRRINAKLLTDERVDLSMLPIADGLTLARKR

SEQ ID NO: 64 - protein, 252 aa, Artificial Sequence
  MxSafC_W96A with tags
SSHHHHHHSSGLVPRGSHMASMTGGQQMGRGSMIHHVELTQSVLQYIRDSSVRDNDILRDLREETSKLPLRTMQIPPEQGQLLSLLVRLIGARKTLEVGVFTGYSTLCAALALPADGRVIACDLSEEAVSIARRYWQRAGVADRIEVRLGDAHHSLEALVGSEHRGTFDLAFIDADKESYDFYYEHALRLVRPGGLIILDNTLWSGKVADPSVVGDPETDSLRRINAKLLTDERVDLSMLPIADGLTLARKR

SEQ ID NO: 65 - protein, 252 aa, Artificial Sequence
  MxSafC_D119P with tags
SSHHHHHHSSGLVPRGSHMASMTGGQQMGRGSMIHHVELTQSVLQYIRDSSVRDNDILRDLREETSKLPLRTMQIPPEQGQLLSLLVRLIGARKTLEVGVFTGYSTLCAALALPADGRVIACDLSEEWVSIARRYWQRAGVADRIEVRLGPAHHSLEALVGSEHRGTFDLAFIDADKESYDFYYEHALRLVRPGGLIILDNTLWSGKVADPSVVGDPETDSLRRINAKLLTDERVDLSMLPIADGLTLARKR

SEQ ID NO: 66 - protein, 252 aa, Artificial Sequence
  MxSafC_T40P with tags
SSHHHHHHSSGLVPRGSHMASMTGGQQMGRGSMIHHVELTQSVLQYIRDSSVRDNDILRDLREETSKLPLRPMQIPPEQGQLLSLLVRLIGARKTLEVGVFTGYSTLCAALALPADGRVIACDLSEEWVSIARRYWQRAGVADRIEVRLGDAHHSLEALVGSEHRGTFDLAFIDADKESYDFYYEHALRLVRPGGLIILDNTLWSGKVADPSVVGDPETDSLRRINAKLLTDERVDLSMLPIADGLTLARKR

SEQ ID NO: 67 - protein, 252 aa, Artificial Sequence
  MxSafC_S173H with tags
SSHHHHHHSSGLVPRGSHMASMTGGQQMGRGSMIHHVELTQSVLQYIRDSSVRDNDILRDLREETSKLPLRTMQIPPEQGQLLSLLVRLIGARKTLEVGVFTGYSTLCAALALPADGRVIACDLSEEWVSIARRYWQRAGVADRIEVRLGDAHHSLEALVGSEHRGTFDLAFIDADKESYDFYYEHALRLVRPGGLIILDNTLWHGKVADPSVVGDPETDSLRRINAKLLTDERVDLSMLPIADGLTLARKR

SEQ ID NO: 68 - protein, 252 aa, Artificial Sequence
  MxSafC_T40P_S173H with tags
SSHHHHHHSSGLVPRGSHMASMTGGQQMGRGSMIHHVELTQSVLQYIRDSSVRDNDILRDLREETSKLPLRPMQIPPEQGQLLSLLVRLIGARKTLEVGVFTGYSTLCAALALPADGRVIACDLSEEWVSIARRYWQRAGVADRIEVRLGDAHHSLEALVGSEHRGTFDLAFIDADKESYDFYYEHALRLVRPGGLIILDNTLWHGKVADPSVVGDPETDSLRRINAKLLTDERVDLSMLPIADGLTLARKR

SEQ ID NO: 69 - protein, 252 aa, Artificial Sequence
  MxSafC_M5 with tags
SSHHHHHHSSGLVPRGSHMASMTGGQQMGRGSMIHHVELTQSVLQYIRDSSVRDNDILRDLREETSKLPLRPMQIPPEQGQLLSLLVRLIGARKTLEVGVFTGYSTLCAALALPADGRVIACDQSEEAVSIARRYWQRAGVADRIEVRLGPAHHSLEALVGSEHRGTFDLAFIDADKESYDFYYEHALRLVRPGGLIILDNTLWHGKVADPSVVGDPETDSLRRINAKLLTDERVDLSMLPIADGLTLARKR

SEQ ID NO: 70 - protein, 220 aa, Artificial Sequence
  MxSafC_L92Q
MIHHVELTQSVLQYIRDSSVRDNDILRDLREETSKLPLRTMQIPPEQGQLLSLLVRLIGARKTLEVGVFTGYSTLCAALALPADGRVIACDQSEEWVSIARRYWQRAGVADRIEVRLGDAHHSLEALVGSEHRGTFDLAFIDADKESYDFYYEHALRLVRPGGLIILDNTLWSGKVADPSVVGDPETDSLRRINAKLLTDERVDLSMLPIADGLTLARKR

SEQ ID NO: 71 - protein, 220 aa, Artificial Sequence
  MxSafC_W96A
MIHHVELTQSVLQYIRDSSVRDNDILRDLREETSKLPLRTMQIPPEQGQLLSLLVRLIGARKTLEVGVFTGYSTLCAALALPADGRVIACDLSEEAVSIARRYWQRAGVADRIEVRLGDAHHSLEALVGSEHRGTFDLAFIDADKESYDFYYEHALRLVRPGGLIILDNTLWSGKVADPSVVGDPETDSLRRINAKLLTDERVDLSMLPIADGLTLARKR

SEQ ID NO: 72 - protein, 220 aa, Artificial Sequence
  MxSafC_D119P
MIHHVELTQSVLQYIRDSSVRDNDILRDLREETSKLPLRTMQIPPEQGQLLSLLVRLIGARKTLEVGVFTGYSTLCAALALPADGRVIACDLSEEWVSIARRYWQRAGVADRIEVRLGPAHHSLEALVGSEHRGTFDLAFIDADKESYDFYYEHALRLVRPGGLIILDNTLWSGKVADPSVVGDPETDSLRRINAKLLTDERVDLSMLPIADGLTLARKR

SEQ ID NO: 73 - protein, 220 aa, Artificial Sequence
  MxSafC_T40P
MIHHVELTQSVLQYIRDSSVRDNDILRDLREETSKLPLRPMQIPPEQGQLLSLLVRLIGARKTLEVGVFTGYSTLCAALALPADGRVIACDLSEEWVSIARRYWQRAGVADRIEVRLGDAHHSLEALVGSEHRGTFDLAFIDADKESYDFYYEHALRLVRPGGLIILDNTLWSGKVADPSVVGDPETDSLRRINAKLLTDERVDLSMLPIADGLTLARKR

SEQ ID NO: 74 - protein, 220 aa, Artificial Sequence
  MxSafC_S173H
MIHHVELTQSVLQYIRDSSVRDNDILRDLREETSKLPLRTMQIPPEQGQLLSLLVRLIGARKTLEVGVFTGYSTLCAALALPADGRVIACDLSEEWVSIARRYWQRAGVADRIEVRLGDAHHSLEALVGSEHRGTFDLAFIDADKESYDFYYEHALRLVRPGGLIILDNTLWHGKVADPSVVGDPETDSLRRINAKLLTDERVDLSMLPIADGLTLARKR

SEQ ID NO: 75 - protein, 220 aa, Artificial Sequence
  MxSafC_T40P_S173H
MIHHVELTQSVLQYIRDSSVRDNDILRDLREETSKLPLRPMQIPPEQGQLLSLLVRLIGARKTLEVGVFTGYSTLCAALALPADGRVIACDLSEEWVSIARRYWQRAGVADRIEVRLGDAHHSLEALVGSEHRGTFDLAFIDADKESYDFYYEHALRLVRPGGLIILDNTLWHGKVADPSVVGDPETDSLRRINAKLLTDERVDLSMLPIADGLTLARKR

SEQ ID NO: 76 - protein, 220 aa, Artificial Sequence
  MxSafC_M5
MIHHVELTQSVLQYIRDSSVRDNDILRDLREETSKLPLRPMQIPPEQGQLLSLLVRLIGARKTLEVGVFTGYSTLCAALALPADGRVIACDQSEEAVSIARRYWQRAGVADRIEVRLGPAHHSLEALVGSEHRGTFDLAFIDADKESYDFYYEHALRLVRPGGLIILDNTLWHGKVADPSVVGDPETDSLRRINAKLLTDERVDLSMLPIADGLTLARKR

(19) United States
(12) Patent Application Publication
(10) Pub. No.: US 2023/0279451 A1
(43) Pub. Date: Sep. 7, 2023

(54) METHODS FOR THE BIOCATALYTICAL MANUFACTURING OF DIHYDROCHALCONES

(71) Applicant: SYMRISE AG, Holzminden (DE)

(72) Inventors: Torsten GEISSLER, Einbeck (DE); Jakob Peter LEY, Holzminden (DE); Bastian ZIRPEL, Holzminden (DE)

(21) Appl. No.: 17/763,735

(22) PCT Filed: Sep. 25, 2020
(86) PCT No.: PCT/EP2020/076979

§ 371 Date: Mar. 25, 2022

(30) Foreign Application Priority Data
Sep. 27, 2019 (WO) PCT/EP2019/076265

(51) Int. Cl. C12P 17/04; C12N 9/10; A23L 27/30

(52) U.S. Cl. CPC C12P 17/04; C12N 9/1007; C12N 9/1085; C12Y 205/01006; A23L 27/33

(57) ABSTRACT

The present invention relates to a biocatalytical method for manufacturing of homoeriodictyol dihydrochalcone and/or hesperetin dihydrochalcone by providing at least one first biocatalyst system for the hydroxylation of phloretin and/or its glycosides as well as at least one second biocatalyst for the methylation of 3-hydroxyphloretin. Further disclosed are microorganisms capable of producing such biocatalysts as well as sequences encoding the biocatalysts. Furthermore, the present invention relates to the use of a mixture obtained by a method as disclosed in the present invention and to specific compositions suitable as sweetness enhancers and/or flavouring agents.

### TECHNOLOGICAL FIELD

The present invention relates to a biocatalytical method for manufacturing of homoeriodictyol dihydrochalcone and/or hesperetin dihydrochalcone by providing at least one first biocatalyst system for the hydroxylation of phloretin and/or its glycosides as well as at least one second biocatalyst for the methylation of 3-hydroxyphloretin. Further disclosed are microorganisms capable of producing such biocatalysts as well as sequences encoding the biocatalysts. New mutant enzymes specifically suitable for the above methods are provided as well. Furthermore, the present invention relates to the use of a mixture obtained by a method as disclosed in the present invention and to specific compositions suitable as sweetness enhancers and/or flavouring agents.

### BACKGROUND OF THE INVENTION

Dihydrochalcones are compounds with an increased sweetness potential and are frequently used in various applications to either increase the sweet impression or to mask bittering substances of foodstuffs, pharmaceuticals, beverages or similar finished goods. There is thus a constant need to provide dihydrochalcones as safe food additive and consequently methods to provide said substances in a reliable manner. The manufacturing of homoeriodictyol dihydrochalcone (1) as well as its sweetness enhancing properties are described in WO2007107596A1. Furthermore, mixtures of homoeriodictyol dihydrochalcone (1) with salivation increasing agents in flavouring compositions are described in US20080227867. Also a masking with homoeriodictyol dihydrochalcone (1) of the bitter taste impression of caffeine was described in US20080227867. The manufacturing of (1) was described in WO2007107596A1 as a catalysed aldol reaction with piperidine of 1,4-di-O-benzyolacetophenone with vanillin. In this chemical reaction, the double bond of the obtained chalcone is hydrated with the aid of a Pd/C catalyst. Further methods comprise the usage of protective groups, other bases or reducing agents. All of the described methods cannot be declared as natural manufacturing methods according to EC 1334/2008.

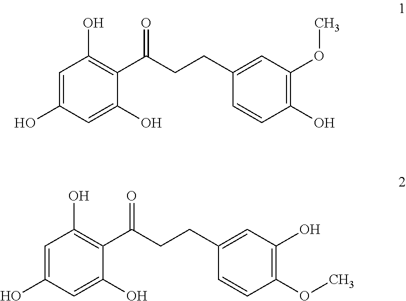 [SMILES: COc1cc(CCC(=O)c2c(O)cc(O)cc2O)ccc1O ; COc1ccc(CCC(=O)c2c(O)cc(O)cc2O)cc1O]

The use and effect of hesperetin dihydrochalcone (2) for modifying unpleasant taste impressions is described in WO 2017186299A1. These characteristics are also described in J. Agric. Food Chem. 1977, 25(4), 763-772 as well as in J. Med. Chem. 1981, 24(4), 408-428. Mixtures of (2) and corn syrup with an increased content of fructose as well as other sweeteners are described in WO2019080990A1. All in all, there are no mixtures of (1) and (2) disclosed in the state of the art.

WO2007107596A1 discloses 4-hydroxychalcones for the improvement of sweet taste impression, wherein the 4-hydroxy function is described as essential for the sweetness enhancing property of the substance. In this application, the structure of 2 is not explicitly disclosed, but a Markush formula implicitly disclosing the structure of 2 is described. Moreover, the effect of structure 2 is not supported or disclosed in the examples.

Hesperetin dihydrochalcone (2) can be manufactured by an acid hydrolysis of Neohesperidin dihydrochalcone which is described in WO2019080990A1. Furthermore, (2) can be manufactured by dissolution of Hesperetin in 10 wt.-% aqueous KOH solution and subsequent reduction with hydrogen with aid of a Pd/C catalyst. The usage of protective groups, other bases or reducing agents as well as the possibility of an acid catalysed aldol reaction is well known in the art. All known methods require organic solvents and can therefore also not be classified as natural manufacturing methods according to EC 1334/2008.

As there is a steadily increasing awareness of consumers towards natural products over the last few years, the labelling as a natural or ecological product is a strong purchasing argument today. It is therefore clear, that a need for naturally manufactured dihydrochalcones which have the same properties as their chemically manufactured pendants is present and rapidly increasing. Notably, an extraction from natural raw materials is not possible due to the unavailability of dihydrochalcones in natural compounds. Therefore, the most promising natural manufacturing method is a biocatalytical approach. This will open the market for application of dihydrochalcones also in finished goods which have an “all-natural”-label.

The object of the present invention is therefore the development of a biocatalytical manufacturing method for homoeriodictyol dihydrochalcone and hesperetin dihydrochalcone and mixtures thereof, which can be classified as produced by a fully natural manufacturing method. Further, it was an object to characterize the resulting products and to improve mixtures and combinations based on homoeriodictyol dihydrochalcone and hesperetin dihydrochalcone with respect to their suitability as flavouring agents and sweetness enhancers by defining precise sensory profiles of compositions comprising these products. Finally, it was an objective to identify and characterize new enzyme variants suitable for enhancing the biocatalytical manufacturing of homoeriodictyol dihydrochalcone and/or hesperetin dihydrochalcone.

### SUMMARY OF THE INVENTION

The above object is solved by providing a biocatalytical method for the manufacturing of homoeriodictyol dihydrochalcone and/or hesperetin dihydrochalcone in a two-step process from phloretin and/or its glycosides using at least one oxidase, at least one reductase and at least one methyltransferase. Further disclosed are possible oxidases and reductases capable of converting phloretin and/or its glycosides into 3-hydroxyphloretin and possible methyltransferases capable of methylating 3-hydroxyphloretin to obtain homoeriodictyol dihydrochalcone and/or hesperetin dihydrochalcone. Further disclosed is the use of a mixture of homoeriodictyol dihydrochalcone and/or hesperetin dihydrochalcone for the use as sweetness enhancer and/or flavouring agent in goods serving the nutrition or the flavour.

In a first aspect of the present invention, a biocatalytical method is provided to manufacture homoeriodictyol dihydrochalcone and/or hesperetin dihydrochalcone using at least one provided biocatalyst system and at least one biocatalyst. First, phloretin is oxidized to obtain 3-hydroxyphloretin by using the at least one first biocatalyst system consisting of at least one oxidase and at least one reductase. The 3-hydroxyphloretin is then reacted with an O-methyltransferase to obtain homoeriodictyol dihydrochalcone and/or hesperetin dihydrochalcone.

In one embodiment of the first aspect, the first and second at least one biocatalyst system or biocatalyst can be provided as an enzyme, a purified enzyme, a whole cell reaction or as a sequence encoding the biocatalyst.

In another embodiment of the first aspect, the second biocatalyst can be an O-methyltransferase.

According to another embodiment of the first aspect, the biocatalyst system or biocatalyst can be purified or partially purified.

In another embodiment of the first aspect, the phloretin and/or its glycosides and/or 3-hydroxyphloretin can be purified or partially purified.

In yet another embodiment of the first aspect, a mixture of homoeriodictyol dihydrochalcone and/or hesperetin dihydrochalcone can be obtained, which can be, according to another embodiment of the first aspect, purified or partially purified.

In a second aspect according to the invention, the mixture of homoeriodictyol dihydrochalcone and/or hesperetin dihydrochalcone can be used as sweetness enhancer and or flavouring agent in goods serving the nutrition or the pleasure.

Further disclosed are organisms that can be used as biocatalysts or as production organisms to produce such biocatalysts and polypeptides especially suited for encoding the biocatalysts disclosed herein.

In yet a further aspect, there is provided an O-methyltransferase suitable as second biocatalyst according to the present disclosure, wherein the O-methyltransferase comprises at least one mutation in comparison to the sequence according to SEQ ID NO: 14, and wherein the O-methyltransferase is selected from the group consisting of SEQ ID NOs: 69 to 76, or a functional fragment thereof, or a sequence having at least 90%, 91%, 92%, 93%, 94%, 95%, 96%, 97%, 98%, or 99% sequence identity to the respective sequence of SEQ ID NOs: 69 to 76, or the functional fragment thereof, or a nucleic acid sequence encoding the O-methyltransferase or the functional fragment thereof.

Finally, in a further aspect, there is provided a composition comprising or consisting of (a) a mixture of homoeriodictyol dihydrochalcone and hesperetin dihydrochalcone in a weight ratio of about 1,000:1 to 1:1,000, or in a weight ratio of about 100:1 to 1:100, preferably about 50:1 to 1:50, more preferably about 10:1 to 1:10, even more preferably about 5:1 to 1:5, and most preferably about 1:1; and (b) and least one of an acid, a further flavour agent, a sweetening agent, and/or water.

Aspects and embodiments of the present invention result from the following detailed description and the examples, the figures, the sequence listing, and the attached claims.

### BRIEF DESCRIPTION OF THE DRAWINGS

FIG. 1: Biotransformation of 3-Hydroxyphloretin with lysed E. coli BL21 (DE 3) cells, which express McPFOMT. Hesperetin dihydrochalcone as well as homoeriodictyol (HED) dihydrochalcone are the products.

FIG. 2: Biotransformation of 3-Hydroxyphloretin with lysed E. coli BL21 (DE 3) cells, which express AtCOMT. Hesperetin dihydrochalcone as well as homoeriodictyol (HED) dihydrochalcone are the products.

FIG. 3: Biotransformation of 3-Hydroxyphloretin with lysed E. coli BL21 (DE 3) cells, which express CrOMT. Hesperetin dihydrochalcone as well as homoeriodictyol (HED) dihydrochalcone are the products.

FIG. 4: Biotransformation of 3-Hydroxyphloretin with lysed E. coli BL21 (DE 3) cells, which express CbMOMT. Hesperetin dihydrochalcone as well as homoeriodictyol (HED) dihydrochalcone are the products.

FIG. 5: Biotransformation of 3-Hydroxyphloretin with lysed E. coli BL21 (DE 3) cells, which express GmSOMT. Hesperetin dihydrochalcone as well as homoeriodictyol (HED) dihydrochalcone are the products.

FIG. 6: Biotransformation of 3-Hydroxyphloretin with lysed E. coli BL21 (DE 3) cells, which express SynOMT. Hesperetin dihydrochalcone as well as homoeriodictyol (HED) dihydrochalcone are the products.

FIG. 7: Cultivation of PPS-9010_CH3H_ATR1 with phloretin. 3-Hydroxyphloretin is the product.

FIG. 8: Incubation of lysed PPS-9010_SAM_MxSafC cells with 3-hydroxyphloretin. The lysate was incubated for 24 hours at 25° C. with 3 mM 3-hydroxyphlortein, 3 mM S-Adenosylmethionin and 0.67 mM MgCl₂. Hesperetin dihydrochalcone as well as homoeriodictyol dihydrochalcone are the products.

FIG. 9: Incubation of lysed PPS-9010_SAM_PsOMT cells with 3-hydroxyphloretin. The lysate was incubated for 24 hours at 25° C. with 3 mM 3-hydroxyphlortein, 3 mM S-Adenosylmethionin and 0.67 mM MgCl₂. Hesperetin dihydrochalcone as well as homoeriodictyol dihydrochalcone are the products.

FIG. 10: Biotransformation of 3-Hydroxyphloretin with lysed E. coli BL21 (DE 3) cells, which express MxSafC (wild-type (wt), cf. SEQ ID NOs: 13, 14, 55), or specific variants or mutants thereof (cf. SEQ ID NOs: 56 to 76). Hesperetin dihydrochalcone as well as homoeriodictyol (HED) dihydrochalcone are the products. Conversion of 3-hyroxyphloretin (30HP) shown in light grey, product specificity towards hesperetin dihydrochalcone is shown in dark grey.

### BRIEF DESCRIPTION OF THE SEQUENCES

SEQ ID NO: 1: Artificial nucleic acid sequence which encodes a variant of a glycerol aldehyde-3-phosphate promoter variant.
SEQ ID NO: 2: Artificial nucleic acid sequence which encodes a variant of a glycerol aldehyde-3-phosphate promoter variant.
SEQ ID NO: 3: Artificial nucleic acid sequence which encodes a resistance gene against bleomycine.
SEQ ID NO: 4: Artificial amino acid sequence which encodes a resistance protein against bleomycine.
SEQ ID NO: 5: Artificial nucleic acid sequence which encodes an aminoglycoside phosphotransferase.
SEQ ID NO: 6: Artificial amino acid sequence which encodes an aminoglycoside phosphotransferase.
SEQ ID NO: 7: describes a nucleic acid sequence from Arabidopsis thaliana encoding a NADPH cytochrome P450 reductase 1.
SEQ ID NO: 8: describes an amino acid sequence from Arabidopsis thaliana encoding a NADPH cytochrome P450 reductase 1.
SEQ ID NO: 9: describes a nucleic acid sequence from Cosmos sulphureus encoding a chalcone-3-hydroxylase.
SEQ ID NO: 10: describes an amino acid sequence from Cosmos sulphureus encoding a chalcone-3-hydroxylase.
SEQ ID NO: 11: describes a nucleic acid sequence from Saccharomyces cerevisiae encoding a S-adenosylmethionine synthase.
SEQ ID NO: 12: describes an amino acid sequence from Saccharomyces cerevisiae encoding a S-adenosylmethionine synthase.
SEQ ID NO: 13: describes a nucleic acid sequence from Myxococcus xanthus encoding an O-methyltransferase.
SEQ ID NO: 14: describes an amino acid sequence from Myxococcus xanthus encoding an O-methyltransferase.
   Reference sequence for numbering of MxSafC mutant positions (cf. SEQ ID NOs: 56 to 76 below).
SEQ ID NO: 15: describes a nucleic acid sequence from Pinus sylvestris encoding an O-methyltransferase.
SEQ ID NO: 16: describes an amino acid sequence from Pinus sylvestris encoding an O-methyltransferase.
SEQ ID NO: 17: Artificial nucleic acid sequence encoding a forward primer.
SEQ ID NO: 18: Artificial nucleic acid sequence encoding a reverse primer.
SEQ ID NO: 19: Artificial nucleic acid sequence encoding a forward primer.
SEQ ID NO: 20: Artificial nucleic acid sequence encoding a reverse primer.
SEQ ID NO: 21: Artificial nucleic acid sequence encoding a forward primer.
SEQ ID NO: 22: Artificial nucleic acid sequence encoding a reverse primer.
SEQ ID NO: 23: Artificial nucleic acid sequence encoding a forward primer.
SEQ ID NO: 24: Artificial nucleic acid sequence encoding a reverse primer.
SEQ ID NO: 25: Artificial nucleic acid sequence encoding a forward primer.
SEQ ID NO: 26: Artificial nucleic acid sequence encoding a reverse primer.
SEQ ID NO: 27: Artificial nucleic acid sequence encoding a forward primer.
SEQ ID NO: 28: Artificial nucleic acid sequence encoding a reverse primer.
SEQ ID NO: 29: Artificial nucleic acid sequence encoding a forward primer.
SEQ ID NO: 30: Artificial nucleic acid sequence encoding a reverse primer.
SEQ ID NO: 31: Artificial nucleic acid sequence encoding a forward primer.
SEQ ID NO: 32: Artificial nucleic acid sequence encoding a reverse primer.
SEQ ID NO: 33: Artificial nucleic acid sequence encoding a forward primer.
SEQ ID NO: 34: Artificial nucleic acid sequence encoding a reverse primer
SEQ ID NO: 35: Artificial nucleic acid sequence encoding a forward primer.
SEQ ID NO: 36: Artificial nucleic acid sequence encoding a reverse primer.
SEQ ID NO: 37: Nucleic acid sequence from Bacillus subtilis encoding a S-adenosylmethionine synthase.
SEQ ID NO: 38: Amino acid sequence from Bacillus subtilis encoding a S-adenosylmethionine synthase.
SEQ ID NO: 39: Nucleic acid sequence from Bacillus subtilis encoding the 1317V mutant of a S-adenosylmethionine synthase.
SEQ ID NO: 40: Amino acid sequence from Bacillus subtilis encoding the 1317V mutant of a S-adenosylmethionine synthase.
SEQ ID NO: 41: Nucleic acid sequence from Escherichia coli encoding a S-adenosylmethionine synthase.
SEQ ID NO: 42: Amino acid sequence from Escherichia coli encoding a S-adenosylmethionine synthase.
SEQ ID NO: 43: Nucleic acid sequence from Streptomyces spectabilis encoding a S-adenosylmethionine synthase.
SEQ ID NO: 44: Nucleic acid sequence from Streptomyces spectabilis encoding a S-adenosylmethionine synthase.
SEQ ID NO: 45: Nucleic acid sequence from Saccharomyces cerevisiae encoding a Glucose-6-phosphate dehydrogenase
SEQ ID NO: 46: Amino acid sequence from Saccharomyces cerevisiae encoding a Glucose-6-phosphate dehydrogenase
SEQ ID NO: 47: Nucleic acid sequence from Komagataella phaffii encoding a Glucose-6-phosphate dehydrogenase
SEQ ID NO: 48: Amino acid sequence from Komagataella phaffii encoding a Glucose-6-phosphate dehydrogenase
SEQ ID NO: 49: Artificial nucleic acid sequence encoding a forward primer.
SEQ ID NO: 50: Artificial nucleic acid sequence encoding a reverse primer.
SEQ ID NO: 51: Artificial nucleic acid sequence which encodes a resistance gene against hygromycine.
SEQ ID NO: 52: Artificial amino acid sequence which encodes a resistance protein against hygromycine.
SEQ ID NO: 53: Artificial nucleic acid sequence encoding a forward primer.
SEQ ID NO: 54: Artificial nucleic acid sequence encoding a reverse primer.
SEQ ID NO: 55: Artificial nucleic acid sequence encoding a O-methyltransferase from Myxococcus xanthus (MxSafC) including tags (including inter alia N-terminal HIS-tag and linker, wherein these elements are not calculated when reference will be made below to the position of a mutation in MxSafC. SEQ ID NO: 14 above serves as reference sequence in that regard).
SEQ ID NO: 56: Artificial nucleic acid sequence encoding a variant of the O-methyltransferase from Myxococcus xanthus (MxSafC_L92Q).
SEQ ID NO: 57: Artificial nucleic acid sequence encoding a variant of the O-methyltransferase from Myxococcus xanthus (MxSafC_W96A).
SEQ ID NO: 58: Artificial nucleic acid sequence encoding a variant of the O-methyltransferase from Myxococcus xanthus (MxSafC_D119P).
SEQ ID NO: 59: Artificial nucleic acid sequence encoding a variant of the O-methyltransferase from Myxococcus xanthus (MxSafC_T40P).
SEQ ID NO: 60: Artificial nucleic acid sequence encoding a variant of the O-methyltransferase from Myxococcus xanthus (MxSafC_S173H).
SEQ ID NO: 61: Artificial nucleic acid sequence encoding a variant of the O-methyltransferase from Myxococcus xanthus (MxSafC_T40P_S173H).
SEQ ID NO: 62: Artificial nucleic acid sequence encoding a variant of the O-methyltransferase from Myxococcus xanthus (MxSafC_M5).
   “M5” herein in this context means a fivefold or quintuple mutant combining mutation T40P/L92Q/W96A/D119P/S173H.
SEQ ID NO: 63: Artificial amino acid sequence of a variant of the O-methyltransferase from Myxococcus xanthus (MxSafC_L92Q) with tags.
SEQ ID NO: 64: Artificial amino acid sequence of a variant of the O-methyltransferase from Myxococcus xanthus (MxSafC_W96A) with tags.
SEQ ID NO: 65: Artificial amino acid sequence of a variant of the O-methyltransferase from Myxococcus xanthus (MxSafC_D119P) with tags.
SEQ ID NO: 66: Artificial amino acid sequence of a variant of the O-methyltransferase from Myxococcus xanthus (MxSafC_T40P) with tags.
SEQ ID NO: 67: Artificial amino acid sequence of a variant of the O-methyltransferase from Myxococcus xanthus (MxSafC_S173H) with tags.
SEQ ID NO: 68: Artificial amino acid sequence of a variant of the O-methyltransferase from Myxococcus xanthus (MxSafC_T40P_S173H) with tags.
SEQ ID NO: 69: Artificial amino acid sequence of a variant of the O-methyltransferase from Myxococcus xanthus (MxSafC_M5) with tags.
SEQ ID NO: 70: Artificial amino acid sequence of a variant of the O-methyltransferase from Myxococcus xanthus (MxSafC_L92Q).
SEQ ID NO: 71: Artificial amino acid sequence of a variant of the O-methyltransferase from Myxococcus xanthus (MxSafC_W96A).
SEQ ID NO: 72: Artificial amino acid sequence of a variant of the O-methyltransferase from Myxococcus xanthus (MxSafC_D119P).
SEQ ID NO: 73: Artificial amino acid sequence of a variant of the O-methyltransferase from Myxococcus xanthus (MxSafC_T40P).
SEQ ID NO: 74: Artificial amino acid sequence of a variant of the O-methyltransferase from Myxococcus xanthus (MxSafC_S173H).
SEQ ID NO: 75: Artificial amino acid sequence of a variant of the O-methyltransferase from Myxococcus xanthus (MxSafC_T40P_S173H).
SEQ ID NO: 76: Artificial amino acid sequence of a variant of the O-methyltransferase from Myxococcus xanthus (MxSafC_M5).

### DETAILED DESCRIPTION

To satisfy the need of providing dihydrochalcones fully manufactures by biocatalytic means relying on a suitable combination of enzymes and the cognate substrates, the present inventors designed a pathway through metabolic engineering and provided suitable enzymes and variants thereof to produce relevant dihydrochalcones starting from phloretin and its glycosides as educts.

According to a first aspect of the present invention, a method for the biocatalytical manufacturing of homoeriodictyol dihydrochalcone and/or hesperetin dihydrochalcone, comprising or consisting of the following steps, may be provided. In a first step (i), at least one first biocatalyst system comprising at least one oxidase or a sequence encoding the same can be provided along with at least one reductase or a sequence encoding the same. In the second step, the method may involve (ii) contacting the at least one first biocatalyst system with phloretin and/or its glycosides and incubating the mixture to (iii) obtain 3-hydroxyphloretin. In step (iv), at least a second biocatalyst can be provided and optionally also at least one methyl group donor, wherein the at least second biocatalyst provided in step (iv) can be contacted in step (v) of the method according to invention with the 3-hydroxyphloretin obtained in step (iii) and optionally with the at least one methyl group donor provided in step (iv) and incubate the mixture to obtain in step (vi) homoeriodictyol dihydrochalcone and/or hesperetin dihydrochalcone.

It was surprisingly found, that with the method according to the invention homoeriodictyol dihydrochalcone and hesperetin dihydrochalcone can be manufactured biocatalytically and functional products in high yields can be obtained. This has some particular advantages over the chemical synthesis disclosed in the state of the art, such as the possibility to declare the product as manufactured by an all-natural process. Furthermore, this method is not based on a high purity of educts, also semi-finished goods and raw educts can be used for manufacturing. A high stereo-selectivity can be achieved only by using enzymes, which is a major advantage over a chemical process. Finally, no addition of harsh chemical substances for chemically catalysing certain reaction steps are needed. All in all, this new biocatalytical method opens the way for manufacturing homoeriodictyol dihydrochalcone and hesperetin dihydrochalcone in an all-natural way and these products can therefore be declared as natural according to EC 1334/2008.

In the context of the present invention, the term biocatalyst means an organism or a catalyst originating from an organism, which is able to catalyse the desired reaction. In this context, at least one biocatalyst catalyses each the oxidation and reduction reaction as well as the methylation of the obtained 3-hydroxyphloretin. Therefore, the biocatalyst may be an enzyme, optionally in purified form, or it may imply an organism comprising at least one enzyme or a sequence encoding the same.

In the context of the present invention, a biocatalyst system comprising at least one oxidase and at least one reductase can be present in the same form or in different forms. In one embodiment of the present invention, both of the at least one enzymes are expressed in the same microorganism. In another embodiment, the biocatalyst system comprises at least two microorganisms each expressing one of the respective enzymes.

According to another embodiment, the biocatalyst system comprises at least two purified or partially purified enzymes or of at least one enzyme expressed in a microorganism and at least one purified or partially purified enzyme.

In yet another embodiment, the at least one oxidase and/or the at least one reductase are present in at least one cell lysate, wherein the term cell lysate describes a microorganism which was subjected to mechanical or chemical treatment after fermentation and which is not viable anymore. In a preferred embodiment, the at least one enzyme of a biocatalyst system may also be produced under the control of a secretory signal so that the enzyme will be secreted by the host cell and the enzyme(s) can be easily retrieved for the cell culture supernatant.

In another embodiment, the at least one oxidase and at least one reductase are present in at least two cell lysates which are pooled together before starting step ii) of the method according to the invention.

The cultivation, isolation, and purification of a recombinant microorganism or fungus or a protein or enzyme encoded by a nucleic acid sequence according to the disclosure of the present invention are known to the person skilled in the art.

The at least one oxidase provided in the biocatalyst system in step i) is mandatory for catalysing the oxidation of phloretin and/or its glycosides, wherein the at least one reductase provided in step i) is mandatory for reducing the oxidized phloretin and/or its oxidized glycosides and therefore obtaining the 3-hydroxyphloretin. It is especially advantageous to use a biocatalyst system, as both reactions can happen simultaneously in comparison to a chemical catalyst.

In one embodiment of the first aspect of the present invention, the glycosides of phloretin can be selected from the group consisting of phloridzine, sieboldin, trilobatin, naringin dihydrochalcone and phloretin-4′-O-glucoside.

Suitable reaction conditions such as buffers, additives, temperature and pH conditions, suitable co-factors, and optionally further proteins can easily be determined by a person skilled in the art with knowledge of the enzymes required therefore, said enzymes also determining the selection of the reaction conditions, according to any aspect or embodiment of the present disclosure.

According to a preferred embodiment of the first aspect of the invention, the first and second of the at least one biocatalyst or biocatalyst system is or are provided as/in at least one of an enzyme, a purified enzyme, a cell lysate, a whole cell reaction or as a sequence encoding the biocatalyst, or a combination thereof.

In context of the present invention, a purified enzyme or partially purified enzyme means the processing of a biotechnological manufactured enzyme to decrease the by-products. This can be done with different separation methods well-known in the art, e.g. chromatography, including affinity chromatography, hydrophobic interaction chromatography, size exclusion chromatography, and the like, precipitation, membrane filtration, centrifugation, crystallisation or sedimentation. A purified enzyme hereby relates to a total content of at least 90% (w/v) enzyme in relation to the complete mixture, wherein a partially purified enzyme relates to a total content of maximum 90% (w/v) enzyme in relation to the complete mixture. In another embodiment, it may be preferably to use less purified enzyme mixtures, for example, in case the enzyme can be directly obtained from a cell culture supernatant, or in case a cell lysate may have certain advantages for the subsequent reaction, or in case significant losses of enzyme may be expected during purification. The skilled person can easily determine the content of and the degree of purity of at least one enzyme of interest in a cell culture lysate and/or supernatant of interest and he can easily combine at least one, two, or at least three or several steps of purification to obtain a higher degree of purity, if desired.

In context of the present invention, a whole cell reaction may be a biocatalytical method, wherein no purified or partially purified enzymes or cell lysates are present. It refers to a reaction mixture of at least one type of organism, which is viable and expresses the at least one biocatalyst.

In one embodiment of the first aspect of the present invention, the biocatalyst can be present as a sequence encoding the biocatalyst. This refers to an amino acid sequence and the corresponding nucleic acid sequence or an amino acid sequence encoding the biocatalyst, wherein the sequence needs to be transferred into a microorganism for expressing the corresponding enzyme. In the context of the enzymes and variants as disclosed herein, the term amino acid sequence, polypeptide, and enzyme are used interchangeably.

In yet another embodiment of the first aspect of the present invention, the biocatalyst may be present as or in a combination of an enzyme, a purified enzyme, a cell lysate, a whole cell reaction or as a sequence encoding the biocatalyst.

One embodiment can be a combination of purified or partially purified enzymes. Another embodiment can be a combination of a purified enzyme or a partially purified enzyme and a cell lysate. Yet another embodiment can be a combination of at least two cell lysates. One embodiment can be a combination of a whole cell reaction and at least one purified or partially purified enzyme. Another embodiment can be a combination of two whole cell reactions.

According to another preferred embodiment of the first aspect of the present invention, the at least one second biocatalyst may be an O-methyltransferase or a sequence encoding the same.

An O-methyltransferase catalyses the transfer of a methyl group from a methyl group donor to a methyl group acceptor in a highly stereo-selective manner. In terms of the method according to the present invention, the O-methyltransferase catalyses the transfer of a methyl group from a donor to 3-hydroxyphloretin to from homoeriodictyol dihydrochalcone and/or hesperetin dihydrochalcone. A large number of different O-methyltransferases is well known in the art, e.g. O-methyltranferases from Myxococcus xanthus, Pinus sylvestris, Mesembryanthem crystallinum, Arabidopsis thaliana, Catharanthus roseus, Clarkia breweri and Glycine max. The O-methyltransferase can also be present, according to another embodiment of the invention as an amino acid sequence and its corresponding nucleic acid sequence encoding the same. The sequence is then transformed in a suitable expression system to express the at least one O-methyltransferases.

In yet a further aspect of the present invention, there is provided an O-methyltransferase suitable as second biocatalyst according to the present disclosure, wherein the O-methyltransferase comprises at least one mutation in comparison to the sequence according to SEQ ID NO: 14, and wherein the O-methyltransferase is selected from the group consisting of SEQ ID NOs: 69 to 76, or a functional fragment thereof, or a sequence having at least 90%, 91%, 92%, 93%, 94%, 95%, 96%, 97%, 98%, or 99% sequence identity to the respective sequence of SEQ ID NOs: 69 to 76, or the functional fragment thereof, or a nucleic acid sequence encoding the O-methyltransferase or the functional fragment thereof. A functional fragment as used in this context means a contiguous fragment of the respective MxSafC mutant sequence which is truncated, but still has the same enzymatic specificity as the cognate full-length enzyme. Functional fragments may be combined with tags, or may be used as fusion proteins. In view of the fact that a functional fragment will be sterically less demanding in comparison to the full-length variant, these functional fragments can be useful in certain settings.

To optimize the various methods disclosed herein, certain MxSafC mutants or variants (these terms are used interchangeably herein) were created and tested as disclosed below. Certain mutants with improved properties in comparison to the wild-type MxSafC (SEQ ID NO: 14) could be generated which can be favourably used in the methods as disclosed herein. In view of the fact that these newly identified mutants have an interesting catalytic activity, these mutants can also be used independently of the methods of the present invention as highly active and specific O-methyltransferases specific for 3-hydroxyphloretin and related structures.

In certain embodiments, an O-methyltransferases of SEQ ID NOs: 70 or 71, or a functional fragment thereof may be preferred for a balanced homoeriodictyol dihydrochalcone/hesperetin dihydrochalcone product mixture. In other embodiments, an O-methyltransferases of SEQ ID NOs: 72 to 76, or a functional fragment thereof may be preferred in case high hesperetin dihydrochalcone yields may be of interest. In certain embodiments, an O-methyltransferases of SEQ ID NOs: 73 to 75, or a functional fragment thereof may be preferred in case a high enzymatic activity and/or conversion rate for/of the substrate 3-hydroxyphloretin may be of interest. In certain embodiments, the single mutations of any one of SEQ ID NOs: 70 to 74 may be combined which is other individually (double mutant), or to create triple and quadruple mutants.

In certain embodiments, nucleic acid sequences encoding the variant O-methyltransferases are provided, for example, with SEQ ID NOs: 56 to 62. In view of the fact that these sequences can be codon-optimized, a variation of the respective nucleic acid sequence, or a fragment thereof, is possible within the scope of the present disclosure as long as the relevant nucleic acid sequence encodes an amino acid sequence selected from the group consisting of SEQ ID NOs: 63 to 68 or 69 to 76, or a functional fragment thereof, or a sequence having at least 90%, 91%, 92%, 93%, 94%, 95%, 96%, 97%, 98%, or 99% sequence identity to the respective sequence of SEQ ID NOs:: 63 to 68 or 69 to 76, or the functional fragment thereof.

In another preferred embodiment of the first aspect of the present invention, the at least one first and/or second biocatalyst system can be a purified or partially purified biocatalyst or biocatalyst system. The term purified relates to the same purification level as stated for the enzymes above. A purified biocatalyst is a biocatalyst with >90% (w/v) biocatalyst content in relation to the complete mixture, whereas a partially purified biocatalyst is a biocatalyst with <90% (w/v) biocatalyst content in relation to the complete mixture. The usage of a purified or partially purified biocatalyst is especially advantageous, because a purified or partially purified catalyst is more reaction-specific than a whole cell reaction or a cell lysate, where different metabolic pathways can lead to undesired side-products. The usage of a purified or partially purified biocatalyst can minimize the possible influence of the production of side products.

In yet another preferred embodiment of the present invention, the at least one first biocatalyst system can comprise at least two sequences encoded by an amino acid sequence independently selected from the group consisting of SEQ ID NO: 8 and 10, or a homologue thereof, or a nucleic acid sequence encoding the respective amino acid sequence, or by an amino acid sequence having at least 90%, 91%, 92%, 93%, 94%, 95%, 96%, 97%, 98% or 99% sequence homology to an amino acid sequence according to any one of SEQ ID NO: 8 and SEQ ID NO: 10 or a nucleic acid sequence encoding the respective amino acid sequence, and wherein the at least one second biocatalyst is encoded by an amino acid sequence selected from the group consisting of SEQ ID NO: 14 and SEQ ID NO: 16, or a homologue thereof, or a nucleic acid sequence encoding the respective amino acid sequence, or by an amino acid sequence having at least 90%, 91%, 92%, 93%, 94%, 95%, 96%, 97%, 98% or 99% sequence homology to an amino acid sequence according to any one of SEQ ID NO: 14 and SEQ ID NO: 16 or a nucleic acid sequence encoding the respective amino acid sequence.

SEQ ID NO: 8 describes an amino acid sequence of a NADPH-cytochrome P450 reductase from Arabidopsis thaliana, whereas SEQ ID NO: 10 describes an amino acid sequence of a chalcone-3-hydroxylase from Cosmos sulphureus. SEQ ID NO: 14 describes an O-methyltransferase from Myxococcus xanthus, whereas SEQ ID NO: 16 describes an O-methyltransferase from Pinus sylvestris. The respective sequences are of exemplary nature and may be exchanged by a homologous enzyme or the sequence encoding the same originating from a different organism provided that the respective enzyme has the relevant substrate specificity and catalytic activity as any one of SEQ ID NO: 8, 10, 14, or 16. The skilled person is well aware of the fact that such homologous enzymes exist in different species. A homologous enzyme suitable for the purpose of the present invention can be identified by commonly available in silico tools for sequence comparison, for example the Needleman-Wunsch, the Smith-Waterman, the BLAST or the FASTA algorithm. Further, the skilled person is well aware of the fact that an enzyme may comprise at least one substitution in comparison to a reference sequence as long as the such modified enzyme still comprises the same substrate specificity and catalytic activity.

Furthermore, an enzyme suitable as biocatalyst according to the various aspects and embodiments of the present invention may be a catalytically active domain or fragment of the respective enzyme it is derived from.

Regarding a suitable second biocatalyst, suitable further enzymes and the sequences encoding the same are disclosed in Table 1 under Example 2 below.

Whenever the present disclosure relates to the percentage of identity of nucleic acid or amino acid sequences to each other these values define those values as obtained by using the EMBOSS Water Pairwise Sequence Alignments (nucleotide) programme (https://www.ebi.ac.uk/Tools/psa/_emboss_water/nucleotide.html) nucleic acids or the EMBOSS Water Pairwise Sequence Alignments (protein) programme (https://www.ebi.ac.uk/Tools/psa/emboss_water/) for amino acid sequences. Alignments or sequence comparisons as used herein refer to an alignment over the whole length of two sequences compared to each other. Those tools provided by the European Molecular Biology Laboratory (EMBL) European Bioinformatics Institute (EBI) for local sequence alignments use a modified Smith-Waterman algorithm (see https://www.ebi.ac.uk/Tools/psa/ and Smith, T. F. & Waterman, M. S. “Identification of common molecular subsequences” Journal of Molecular Biology, 1981 147 (1):195-197). When conducting an alignment, the default parameters defined by the EMBL-EBI are used. Those parameters are (i) for amino acid sequences: Matrix=BLOSUM62, gap open penalty=10 and gap extend penalty=0.5 or (ii) for nucleic acid sequences: Matrix=DNAfull, gap open penalty=10 and gap extend penalty=0.5. The skilled person is well aware of the fact that, for example, a sequence encoding a protein can be “codon-optimized” if the respective sequence is to be used in another organism in comparison to the original organism a molecule originates from.

In another preferred embodiment, the at least one first biocatalyst system can additionally comprise at least one dehydrogenase or a sequence encoding the same, preferably a Glucose-6-phosphate dehydrogenase (G6P dehydrogenase) or a sequence encoding the same, wherein the at least one G6P dehydrogenase is encoded by an amino acid sequence selected from the group consisting of SEQ ID NO: 46 and SEQ ID NO: 48 or a homologue thereof, or a nucleic acid sequence encoding the respective amino acid sequence, or by an amino acid sequence having at least 90%, 91%, 92%, 93%, 94%, 95%, 96%, 97%, 98% or 99% sequence homology to an amino acid sequence according to any one of SEQ ID NO: 46 and SEQ ID NO: 48 or a nucleic acid sequence encoding the respective amino acid sequence.

SEQ ID NO: 46 describes a sequence of Glucose-6-phosphate dehydrogenase from Saccharomyces cerevisiae, whereas SEQ ID NO: 48 describes a sequence of Glucose-6-phosphate dehydrogenase from Komagataella phaffii.

The G6P dehydrogenase catalyses transformation of Glucose-6-phosphate to 6-phosphogluconolactone under the formation of NADPH from NADP⁺. As in the reaction of the CYP450 oxidase NADPH is consumed to NADP⁺ it is beneficial to co-express the G6P dehydrogenase to provide sufficient cofactor for the oxidation of phloretin and/or its glycosides towards 3-hydroxyphloretin.

In yet another preferred embodiment of the method according to the invention the at least one oxidase of the first biocatalyst system can be a CYP450 oxidase, and wherein the at least one reductase of the first biocatalyst system can be a CYP450 reductase, preferably wherein the at least one CYP450 oxidase and/or the at least one CYP450 reductase is/are as defined in claim 5.

The CYP450 oxidase and reductase are Cytochrome 450 oxidases and reductases which are present in nearly all of the live on earth. They act as a monooxygenase or monoreductase and catalyses the transfer of one oxygen atom. In terms of the present invention, it is particularly beneficial to use such oxidases as they are ubiquitous available and easily transferrable into a suitable biocatalyst system according to the invention.

According to another preferred embodiment of the first aspect according to the invention the biocatalyst can be produced by or present in a cell selected from the group consisting of Escherichia coli spp., such as E. coli BL21, E. coli MG1655, preferably E. coli W3110, Bacillus spp., such as Bacillus licheniformis, Bacillus subitilis, or Bacillus amyloliquefaciens, Saccharomyces spp., preferably S. cerevesiae, Hansenula or Komagataella spp., such as. K. phaffii and H. polymorpha, preferably K. phaffii, Yarrowia spp. such as Y. lipolytica, Kluyveromyces spp, such as K. lactis.

Methods for breeding and cultivating the recombinant microorganisms, yeasts and fungi according to the present invention and allowing the expression of enzymes according to the present invention and the conversion of the reactants according to the present invention using the disclosed biocatalyst system or biocatalyst are known to the person skilled in the art.

In another preferred embodiment, the incubation in step ii) and iv) can be done done for at least 5, 10, 15, 20, 25 minutes, preferably for at least 30 minutes. In one embodiment of the present invention the incubation time is between 5 and 60 minutes. In another embodiment, the incubation is between 10 and 50 minutes. In yet another embodiment, the incubation time is between 15 and 45 minutes.

In yet another preferred embodiment, the steps i) and ii), or steps i), ii), iv) and v), or steps iv) and v) of the method according to the invention can be conducted simultaneously. In a first embodiment, the step of providing the at least one biocatalytical system can happen together with the contacting of the at least one biocatalytical system with phloretin and/or its glycosides and incubating the mixture. In a second embodiment, the step of providing the at least one biocatalytical system can happen together with the contacting of the at least one biocatalytical system with phloretin and/or its glycosides and the step of providing a second biocatalyst and contacting both biocatalyst with phloretin and/or its glycosides, the product 3-hydroxyphloretin and optionally with the at least one methyl group donor and incubating the mixture to obtain only one reaction mixture. In a third embodiment, the step of providing the at least one second biocatalyst can happen simultaneously together with the step of contacting the second biocatalyst with 3-hydroxyphloretin and optionally the at least one methyl group donor and incubating the mixture.

According to another preferred embodiment of the first aspect the phloretin and/or its glycosides provided in step ii) and/or the 3-hydroxyphloretin obtained in step iii) can be additionally purified or partially purified. Purified refers to a mixture of >90% (w/v) of phloretin and/or its glycosides and/or 3-hydroxyphloretin in relation to the total content of the mixture, whereas a partially purified mixture relates to a mixture of <90% (w/v) of phloretin and/or its glycosides and/or 3-hydroxyphloretin in relation to the total content of the mixture. Suitable purification methods are well known to a person skilled in the art and can be selected from the group consisting of separation by chromatography, rotation vaporisation, spray drying, freeze drying and mechanical separation.

In yet another preferred embodiment, the method according to the invention can comprise adding at least one methyl group donor, and wherein the at least one methyl group donor can be selected from the combination of S-adenosylmethionin and/or methionine and a S-adenosylmethionine synthase (SAM), wherein the S-adenosylmethionine synthase can have an amino acid sequence selected from the group consisting of SEQ ID NO: 12, SEQ ID NO: 38, SEQ ID NO: 40, SEQ ID NO: 42, SEQ ID NO: 44, or a homologue thereof, or a nucleic acid sequence encoding the respective amino acid sequence SEQ ID NO: 12, SEQ ID NO: 38, SEQ ID NO: 40, SEQ ID NO: 42, SEQ ID NO: 44 or by an amino acid sequence having at least 90%, 91%, 92%, 93%, 94%, 95%, 96%, 97%, 98% or 99% sequence homology to an amino acid sequence according to any one of SEQ ID NO: 12, SEQ ID NO: 38, SEQ ID NO: 40, SEQ ID NO: 42, SEQ ID NO: 44 or a nucleic acid sequence encoding the respective amino acid sequence.

SEQ ID NO: 12 describes an amino acid sequence of a S-adenosylmethionine synthase from Saccharomyces cerevisiae, whereas SEQ ID NO: 38 describes an amino acid sequence of a S-adenosylmethionine synthase from Bacillus subtilis. SEQ ID NO: 40 describes an amino acid sequence of a S-adenosylmethionine synthase from the 1317V mutant of Bacillus subtilis, whereas SEQ ID NO: 42 describes an amino acid sequence of a S-adenosylmethionine synthase from Escherichia coli and SEQ ID NO: 44 describes an amino acid sequence of a S-adenosylmethionine synthase from Streptomyces spectabilis.

S-adenosylmethionine synthases (SAMSs) for use according to all considerations of the present invention are those able, due to the substrate specificity and regional selectivity thereof, to catalyze the conversion of ATP and methionine to S-adenosylmethionine. A methyl group donor is every component with a methyl group which can be transferred by a methyltransferase to another component and therefore methylating it.

In another preferred embodiment, the method according to the invention is a method for the biocatalytical manufacturing of a mixture of homoeriodictyol dihydrochalcone and hesperetin dihydrochalcone, wherein step v) of the method according to the invention comprises obtaining a mixture of homoeriodictyol dihydrochalcone and hesperetin dihydrochalcone.

According to a preferred embodiment of first aspect of the invention, the method can comprise an additional step of purifying or partially purifying the obtained homoeriodictyol dihydrochalcone and/or hesperetin dihydrochalcone. Purified refers to a mixture of >90% (w/v) of homoeriodictyol dihydrochalcone and/or hesperetin dihydrochalcone in relation to the total content of the mixture, whereas a partially purified mixture relates to a mixture of <90% (w/v) in relation to the total content of the mixture. Suitable purification methods are well known to a person skilled in the art and can be selected from the group consisting of separation by chromatography, rotation vaporisation, spray drying, freeze drying and mechanical separation.

In a second aspect of the invention, the invention relates to a use of a mixture according to the invention as a sweetness enhancer and/or flavouring agent, preferably wherein the sweetness enhancer and/or flavouring agent is used in finished goods selected from the group consisting of goods intended for nutrition or enjoyment.

In a further embodiment, the mixture according to the invention may be used as a sweetness enhancer and/or flavouring agent in a therapeutic formulation to mask or improve any unfavourable taste of a pharmaceutical product in liquid, gel, or solid form to ease the swallowing and/or uptake of the relevant product or composition by improving its taste.

In yet another aspect, there is provided a composition, wherein the composition may comprise or consist of (a) a mixture of homoeriodictyol dihydrochalcone and hesperetin dihydrochalcone in a weight ratio of about 1,000:1 to 1:1,000, or in a weight ratio of about 100:1 to 1:100, preferably about 50:1 to 1:50, more preferably about 10:1 to 1:10, even more preferably about 5:1 to 1:5, and most preferably about 1:1; and (b) and least one of an acid, a further flavour agent, a sweetening agent, and/or water.

Based on the products as obtainable by the purely biotechnological methods disclosed herein, the further characterization of the sensory profile of these products surprisingly showed that specific mixtures of homoeriodictyol dihydrochalcone and hesperetin dihydrochalcone have a strongly improved effect as taste and sweetness enhancers so that products relying on the specific mixtures can be favourably used in finished consumable goods. In particular, it was found that defined mixtures based on homoeriodictyol dihydrochalcone and hesperetin dihydrochalcone have a strongly improved sensory profile in direct comparison to compositions only comprising hesperetin dihydrochalcone alone, or homoeriodictyol, or homoeriodictyol dihydrochalcone alone.

The ultimate weight ratio of homoeriodictyol dihydrochalcone to hesperetin dihydrochalcone may vary depending on the complexity of the final composition or good. Therefore, in less complex compositions a weight ratio of of about 1,000:1 to 1:1,000, and preferably about 100:1 to 1:100 may be favourable.

In preferred embodiments, the weight ratio of homoeriodictyol dihydrochalcone to hesperetin dihydrochalcone will be in the range of about 50:1 to 1:50, even more preferably about 10:1 to 1:10 or 5:1 to 1:5, and most preferably about 1:1.

It was surprisingly found that a nearly equal mass ratio of 1:1 of homoeriodictyol dihydrochalcone to hesperetin dihydrochalcone may strongly increase the sweetness and taste profile of an aqueous solution comprising these substances, as the resulting mixtures were found to be sweeter and the aroma of vanilla was more pronounced.

In certain embodiments, it may be preferable produce a surplus of either homoeriodictyol dihydrochalcone or hesperetin dihydrochalcone, which can be achieved by balancing the product profile base on the present disclosure.

In another embodiment, the composition comprising or consisting of a mixture of homoeriodictyol dihydrochalcone and hesperetin dihydrochalcone may be combined with an organic acid, including citric acid, tartaric and succinic acid and the like and optionally at least one further sweetening agent. In particular, the addition of an organic acid was found to improve the taste profile, as the resulting mixtures were less sour and astringent in comparison to identical mixtures just using hesperetin dihydrochalcone, in particular if weight ratios of about 10:1 to 1:10 to about 1:1 of homoeriodictyol dihydrochalcone and hesperetin dihydrochalcone were used.

In yet another embodiment, mixture of homoeriodictyol dihydrochalcone and hesperetin dihydrochalcone in the weight ratios identified above may be used together with a further bitter masker, aroma agent, or sweetening agent or any taste improving substance. In one embodiment, the mixture may be combined with a rebaudiosed, for example, rebaudioside A (RebA). It was surprisingly found that the resulting mixtures in weight ratios of about 10:1 to 1:10 to about 1:1 of homoeriodictyol dihydrochalcone and hesperetin dihydrochalcone had a fuller flavour and a richer head in comparison to identical mixtures just using hesperetin dihydrochalcone.

A mixture of sweetness enhancers and/or flavouring agents according to the invention can be used in finished goods intended for nutrition or enjoyment, this can be particularly products such as bakery products (e.g. bread, dry biscuits, cake, other pastries), confectionery (e.g. chocolates, chocolate bar products, other bar products, fruit gum, hard and soft caramel, chewing gum), alcoholic or non-alcoholic drinks (e.g. coffee, tea, wine, drinks containing wine, beer, drinks containing beer, liqueurs, schnapps, brandies, lemonades containing fruit, isotonic drinks, refreshing drinks, nectars, fruit and vegetable juices, fruit and vegetable juice preparations), instant drinks (e.g. instant cocoa drinks, instant tea drinks, instant coffee drinks), meat products (e.g. ham, fresh sausage or raw sausage preparations, flavoured or marinated fresh or salt meat products), eggs or egg products (dry egg, protein, yolk), cereal products (e.g. breakfast cereals, muesli bars, precooked instant rice products), milk products (e.g. milk drinks, milk ice cream, yoghurt, kefir, cream cheese, soft cheese, hard cheese, dry milk powder, whey, butter, buttermilk, partly or completely hydrolysed products containing milk protein), products made of soy protein or other soy bean fractions (e.g. soy milk and products obtained therefrom, compositions containing soy lecithin, fermented products as tofu or tempe or products made therewith), fruit preparations (e.g. jams, fruit ice cream, fruit sauces, fruit fillings), vegetable preparations (e.g. ketchup, sauces, dry vegetables, frozen vegetables, precooked vegetables, boiled down vegetables), snacks (e.g. baked or fried potato chips or potato dough products, extrudates based on corn or peanut), products based on fat and oil or emulsions thereof (e.g. mayonnaise, remoulade, dressings), other finished products and soups (e.g. dry soups, instant soups, precooked soups).

The present invention is further explained by means of the following examples, which are not intended to limit the scope of the present invention, but serve as illustration only.

### EXAMPLES

### Example 1: Transformation of Plasmid DNA in Escherichia coli Cells

The plasmid DNA was transformed into chemically competent Escherichia coli (E. coli) DH5a cells (New England Biolabs, Frankfurt am Main, Germany) in order to propagate the plasmids produced. The plasmid DNA was transformed into chemically competent E. coli BL21(DE3) cells for the production of expression strains.

50 μl aliquots of the corresponding E. coli strain were incubated on ice for 5 minutes. After addition of 1 μl plasmid DNA, the suspension was mixed and incubated on ice for additional 30 minutes. The transformation was performed by incubating the suspension for 45 s at 42° C. in a thermoblock and subsequently on ice for 2 minutes. Then 350 μl SOC Outgrowth Medium (New England Biolabs, Frankfurt am Main, Germany) was added and the cells were incubated for 1 h at 37° C. and 200 rpm. The cell suspension was then spread on LB-agar (Carl Roth GmbH, Karlsruhe, Germany) with the respective antibiotic and the plate incubated for 16 h at 37° C. The cells were then incubated for 1 h at 37° C. and 200 rpm.

### Example 2: Generating the E. coli Expression Strains

The following expression vectors with O-methyltransferases from different organisms are transformed as described in Example 1 in E. coli BL21 (DE 3) cells.

TABLE 1

| Plasmid | Organism |
|---|---|
| pET28a_McPFOMT | Mesembryanthem crystallinum |
| pQE30_AtCOMT | Arabidopsis thaliana |
| pET28a_CrOMT | Catharanthus roseus |
| pET28a_CbMOMT | Clarkia breweri |
| pET28a_GmSOMT | Glycine max |
| pQE30_SynOMT | Synechocystis sp. PCC 6803 |
| pET28a_MxSafC (wt) | Myxooccus xanthus |
| pET28a_MxSafC_L92Q | Myxooccus xanthus |
| pET28a_MxSafC_W96A | Myxooccus xanthus |
| pET28a_MxSafC_D119P | Myxooccus xanthus |
| pET28a_MxSafC_T40P | Myxooccus xanthus |
| pET28a_MxSafC_S173H | Myxooccus xanthus |
| pET28a_MxSafC_T40P_S173H | Myxooccus xanthus |
| pET28a_MxSafC_M5 | Myxooccus xanthus |

McPFOMT and the sequence encoding the same correspond to SEQ ID NO: 24 and 4 as disclosed in EP3050971. AtCOMT and the sequence encoding the same correspond to SEQ ID NO: 23 and 3 as disclosed in EP 3050974. CrOMT and the sequence encoding the same corresponds to SEQ ID NO: 36 and 16 as disclosed in EP3050971. CbMOMT and the sequence encoding the same relates to SEQ ID NO: 27 and 7 as disclosed in EP3050971. GmSOMT and the sequence encoding the same relate to SEQ ID NO: 25 and as disclosed in EP3050971. SynOMT and the sequence encoding the same correspond to SEQ ID NO: 39 and 19 as disclosed in EP3050971.

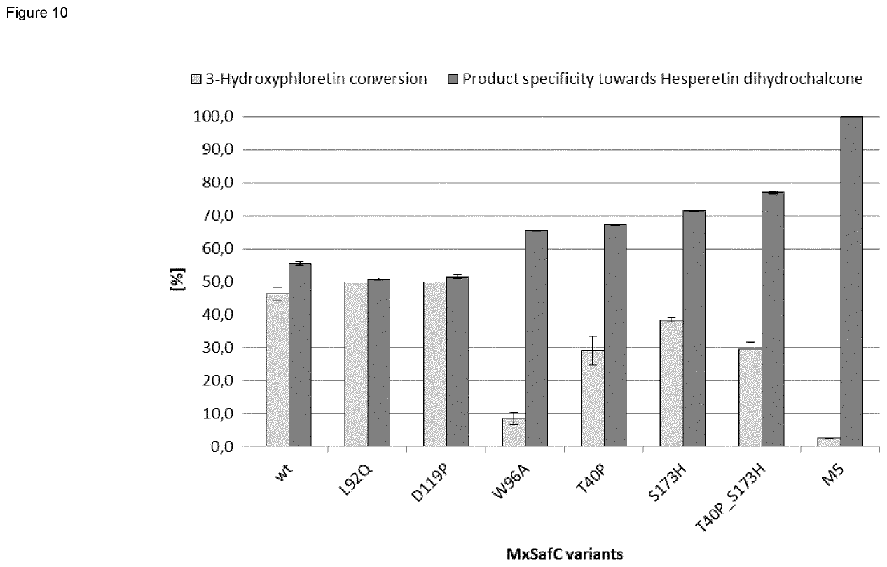

MxSafC mutant variants correspond to SEQ ID NOs: 56 to SEQ ID NO: 76. These were synthesized (BioCat GmbH, Heidelberg, Germany) and cloned into pET28a between NcoI and HindIII restriction sites, respectively. SEQ ID NOs: 13, 14 and 55 show the respective wild-type sequence. By artificial design, certain mutants were created to optimize the activity and/or specificity of the MxSafC enzyme. Interesting single, double and quintuple mutants could be identified, as shown in detail in the results in FIG. 10.

First, various positions within MxSafC (SEQ ID NO: 14) were randomly mutated. All mutants were characterized and checked for activity. In a second round, targeted mutations and combinations thereof were tested in an iterative manner to identify suitable mutants for commercial purposes that have a good activity and conversion rate, but at the same time a good or even improved product specificity. Indeed, certain variants could be identified that fulfill these needs.

Notably, all mutants had a great product specificity towards hesperetin dihydrochalcone. Interestingly, for the L92Q and the D119P variant, the specificity for homoeriodictyol dihydrochalcone and hesperetin dihydrochalcone are nearly balanced, which may be preferably for certain assays where a balanced product mixture is desired. In case product specificity for hesperetin dihydrochalcone is of utmost importance, the W96A, T40P, S173H, T40P/S173H, and the M5 quintuple mutant seem to be very promising, as all of these mutants or variants performed better than the wild-type regarding the relevant characteristic of specificity. Regarding enzyme activity (light grey bars in FIG. 10), all mutants/variants were active. The T40P and the S173H mutant were identified as particularly favorable in first experiments. Therefore, an additional double mutant (T40P/S173H) was created, which showed both: a good specificity as well as a technically reasonable activity. The latter mutants may thus be preferable in case high yields per enzyme unit used may be of interest.

### Example 3: Cultivation of E. coli Cells and Biotransformation

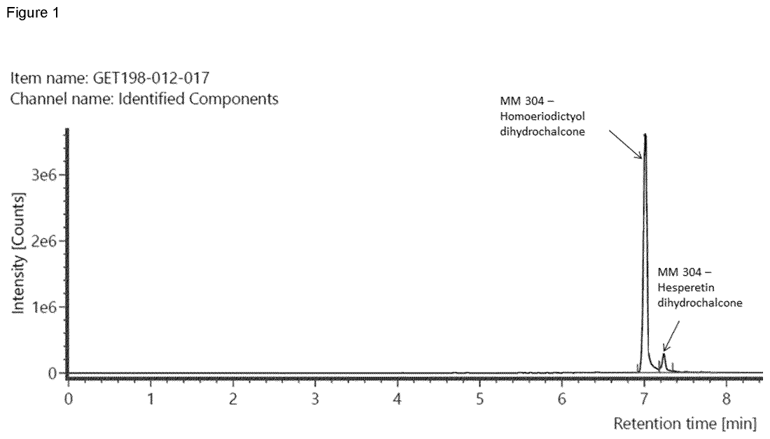

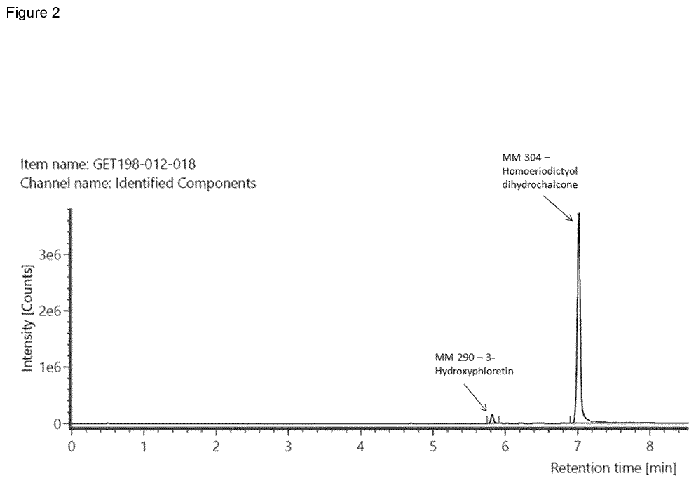

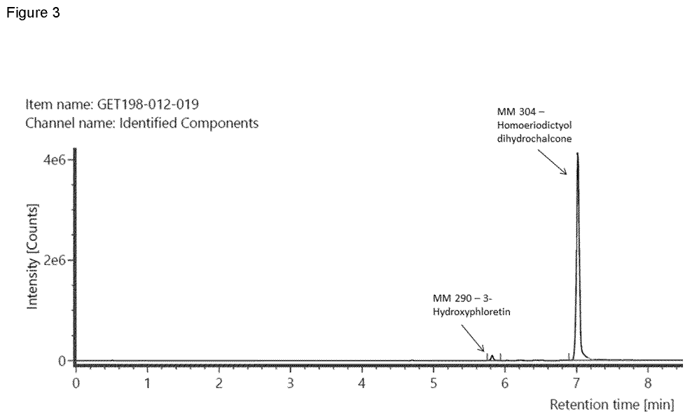

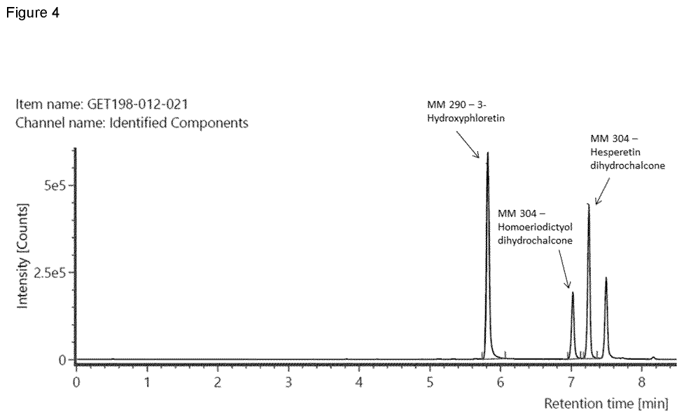

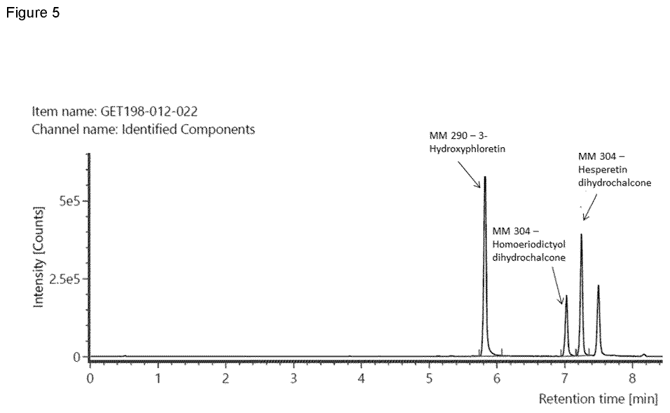

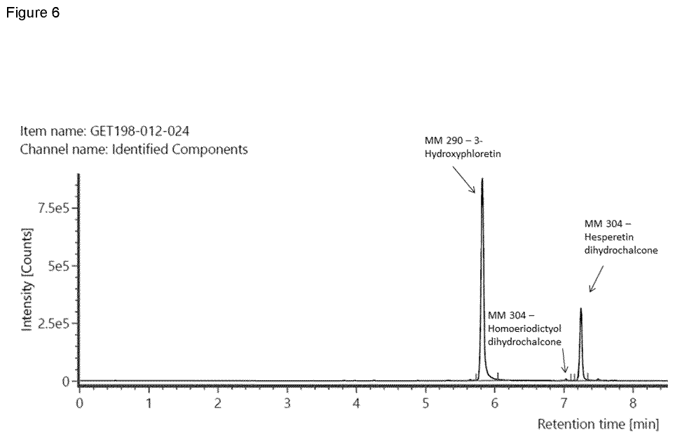

30 E. coli BL21(DE3) cells, each containing a plasmid from Table 1, were used to inoculate 5 ml of LB medium (Carl Roth GmbH, Karlsruhe, Germany) with the corresponding antibiotic. After incubation for 16 h (37° C., 200 rpm), 20 ml TB medium (Carl Roth GmbH, Karlsruhe, Germany) were inoculated with an OD600 of 0.1 from these cultures. These main cultures were incubated (37° C., 200 rpm) until an OD600 of 0.5-0.8 was achieved. After addition of 1 mM isopropyl-β-D-thiogalactopyranoside, the cultures were incubated for a further 16 h (22° C., 200 rpm). The main culture was centrifuged (10 min, 10,000 rpm), the pelleted cells were decomposed using the B-PER protein extraction reagent (Thermo Fisher Scientific, Bonn, Germany) according to the manufacturers specifications. After additional centrifugation (10 min, 14,000 rpm) the supernatant was mixed with 3 mM 3-hydroxyphloretin, 3 mM S-adenosylmethionine, 0.1 mM MgCl2. The reaction mixture was incubated at 25° C. for 24 hours. After stopping the assay with 20% trichloroacetic acid (5.7 final concentration) the sample was centrifuged and the supernatant used for LC-MS analysis. The results of the biocatalysis are displayed in FIGS. 1 to 6 and 10.

### Example 4: Generating the Expression Vectors for Komagataella phaffii

The sequence SEQ ID NO: 1 was synthesized (BioCat GmbH, Heidelberg, Germany). The SEQ ID NO: 2 of pPICZalphaA (BioCat GmbH, Heidelberg, Germany) was exchanged with SEQ ID NO: 1 to obtain the vector pG1Za_EV. Therefore, pPICZalphaA with SEQ ID NO: 17 and SEQ ID NO: 18 as well as SEQ ID NO: 1 with SEQ ID NO: 19 and SEQ ID NO: 20 were amplified by polymerase chain reaction (PCR) according to common practice known to experts, the reaction solutions were mixed in a ratio of 1:1 and 1.5 μl of the mixture was transformed into E. coli DH5a after 1 h incubation at 37° C. as described in Example 1.

The SEQ ID NO: 3, which encodes SEQ ID NO: 4, of vector pG1Za_EV was replaced with SEQ ID NO: 5, which encodes SEQ ID NO: 6, of vector pPIC9K (Biocat GmbH, Heidelberg, Germany) to obtain vector pG1Ga_EV. Vector pG1Za_EV with SEQ ID NO: 21 and SEQ ID NO: 22 as well as SEQ ID NO: 5 with SEQ ID NO: 23 and SEQ ID NO: 24 were amplified by polymerase chain reaction (PCR) according to common practice known to experts, the reaction solutions were mixed in a ratio of 1:1 and 1.5 μl of the mixture was transformed into E. coli DH5a after 1 h incubation at 37° C. as described in Example 1.

The sequence SEQ ID NO: 51 was synthesized (BioCat GmbH, Heidelberg, Germany). The SEQ ID NO: 3, which encodes SEQ ID NO: 4, of vector pG1Za_EV was replaced with SEQ ID NO: 51, which encodes SEQ ID NO: 52, to obtain vector pG1Ha_EV. Vector pG1Za_EV with SEQ ID NO: 21 and SEQ ID NO: 22 as well as SEQ ID NO: 51 with SEQ ID NO: 53 and SEQ ID NO: 54 were amplified by polymerase chain reaction (PCR) according to common practice known to experts, the reaction solutions were mixed in a ratio of 1:1 and 1.5 μl of the mixture was transformed into E. coli DH5a after 1 h incubation at 37° C. as described in Example 1. The gene sequence SEQ ID NO: 7 coding for SEQ ID NO: 8 was synthesized (BioCat, Heidelberg, Germany) and cloned in pG1Za_EV between SEQ ID NO: 1 and AOX1 terminator to obtain vector pG1Z_ATR1. Vector pG1Za_EV with SEQ ID NO: 25 and SEQ ID NO: 26 as well as SEQ ID NO: 7 with SEQ ID NO: 27 and SEQ ID NO: 28 were amplified by polymerase chain reaction (PCR) according to common practice known to experts, the reaction solutions were mixed in a ratio of 1:1 and 1.5 μl of the mixture was transformed into E. coli DH5a after 1 h incubation at 37° C. as described in Example 1.

The gene sequence SEQ ID NO: 9 coding for SEQ ID NO: 10 was synthesized (BioCat, Heidelberg, Germany) and cloned in pG1Ga_EV between SEQ ID NO: 1 and AOX1 terminator to obtain vector pG1G_CH3H. Vector pG1Ga_EV with SEQ ID NO: 25 and SEQ ID NO: 26 as well as SEQ ID NO: 9 with SEQ ID NO: 29 and SEQ ID NO: 30 were amplified by polymerase chain reaction (PCR) according to common practice known to experts, the reaction solutions were mixed in a ratio of 1:1 and 1.5 μl of the mixture was transformed into E. coli DH5a after 1 h incubation at 37° C. as described in Example 1.

The gene sequence SEQ ID NO: 11, which encodes SEQ ID NO: 12, was synthesized (BioCat, Heidelberg, Germany) and cloned in pG1Za_EV between SEQ ID NO: 1 and AOX1 terminator to obtain vector pG1Z_SAM2. Vector pG1Za_EV with SEQ ID NO: 25 and SEQ ID NO: 26 as well as SEQ ID NO: 11 with SEQ ID NO: 31 and SEQ ID NO: 32 were amplified by polymerase chain reaction (PCR) according to common practice known to experts, the reaction solutions were mixed in a ratio of 1:1 and 1.5 μl of the mixture was transformed into E. coli DH5a after 1 h incubation at 37° C. as described in Example 1.

The gene sequence SEQ ID NO: 13, which encodes SEQ ID NO: 14, was synthesized (BioCat, Heidelberg, Germany) and cloned in pG1Ga_EV between SEQ ID NO: 1 and AOX1 terminator to obtain vector pG1G_MxSafC. Vector pG1Ga_EV with SEQ ID NO: 25 and SEQ ID NO: 26 as well as SEQ ID NO: 13 with SEQ ID NO: 33 and SEQ ID NO: 34 were amplified by polymerase chain reaction (PCR) according to common practice known to experts, the reaction solutions were mixed in a ratio of 1:1 and 1.5 μl of the mixture was transformed into E. coli DH5a after 1 h incubation at 37° C. as described in Example 1.

The gene sequence SEQ ID NO:15 coding for SEQ ID NO:16 was synthesized (BioCat, Heidelberg, Germany) and cloned in pG1Ga_EV between SEQ ID NO:1 and AOX1 terminator to obtain vector pG1G_PsOMT. Vector pG1Ga_EV with SEQ ID NO:25 and SEQ ID NO:26 as well as SEQ ID NO:15 with SEQ ID NO:35 and SEQ ID NO:36 were amplified by polymerase chain reaction (PCR) according to common practice known to experts, the reaction solutions were mixed in a ratio of 1:1 and 1.5 μl of the mixture was transformed into E. coli DH5a after 1 h incubation at 37° C. as described in Example 1.

The gene sequence SEQ ID NO:45 coding for SEQ ID NO:46 was synthesized (BioCat, Heidelberg, Germany) and cloned in pG1Ga_EV between SEQ ID NO:1 and AOX1 terminator to obtain vector pG1H_G6PDH. Vector pG1 Ha_EV with SEQ ID NO:25 and SEQ ID NO:26 as well as SEQ ID NO:45 with SEQ ID NO:49 and SEQ ID NO:50 were amplified by polymerase chain reaction (PCR) according to common practice known to experts, the reaction solutions were mixed in a ratio of 1:1 and 1.5 μl of the mixture was transformed into E. coli DH5a after 1 h incubation at 37° C. as described in Example 1.

### Example 5: Transformation of Linearized Plasmid DNA in Komagataella phaffii Cells

Electrically competent cells of the respective stem were created (Lin-Cereghino et al., 2005) and transformed with the corresponding linearized vector. 200 ng/μl of the vector were digested with AvrII and 4 μl reaction solution was transformed with 40 μl aliquot of electrocompetent cells at 1.8 kV. After addition of 500 μl 1 M sorbitol and 500 μl YPD (10 g/I yeast extract, 20 g/I peptone, 10 g/I glucose, 0.67 g/I yeast nitrogen base with ammonium sulfate, 100 mM phosphate buffer pH 6.5, 10 g/I methionine) the cells were incubated (30° C., 200 rpm, 2 h) and 50 μl were plated on YPD agar plates with the corresponding antibiotic (Zeocin: 100 μg/ml, Geneticin: 400 μg/ml). After incubation for 48 h at 30° C. the transformants were selected for cultivation.

### Example 6: Generation of Komagataella phaffii Expression Strains

The strain PPS-9010 was acquired from ATUM (Newark, Calif.).

Strain PPS-9010 was transformed with linearized vector pG1G_CH3H. A selected transformant was subsequently transformed with linearized vector pG1Z_ATR1 to obtain strain PPS-9010_CH3H_ATR1. A selected transformant of strain PPS-9010_CH3H_ATR1 was subsequently transformed with linearized vector pG1H_G6PDH to obtain strain PPS-9010_CH3H_ATR1_G6PDH. Strain PPS-9010 was transformed with linearized vector pG1Z_SAM2. A selected transformant was subsequently transformed with linearized vector pG1G_MxSafC or pG1G_PsOMT to obtain strain PPS-9010_SAM_MxSafC or PPS-9010_SAM_PsOMT respectively.

### Example 7: Cultivation of Komagataella phaffii Cells and Biotransformation

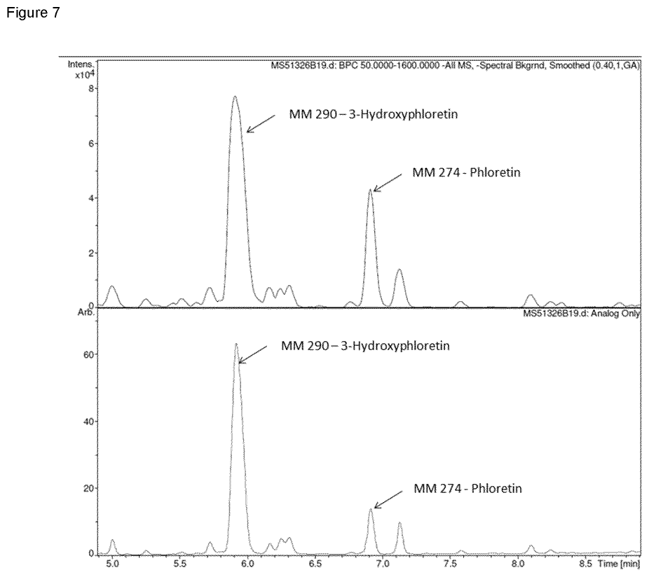

Cells of PPS-9010_CH3H_ATR1 were used to inoculate 10 ml BMGYM medium (10 g/I yeast extract, 20 g/I peptone). After incubation for 16 h (30° C., 200 rpm) another 25 ml BMGYM from the described previous culture was inoculated to OD600=0.2. After incubation (30° C., 200 rpm) up to an OD600 of 0.8-1.0 400 ppm phloretin were added. After incubation for 20 h at 25° C., 1% glycerol and 400 ppm phloretin were added and further incubation for 24 h was performed. The culture was then mixed with 20% trichloroacetic acid, centrifuged and the supernatant used for LC-MS analysis. The result of the biocatalysis is depicted in FIG. 7.

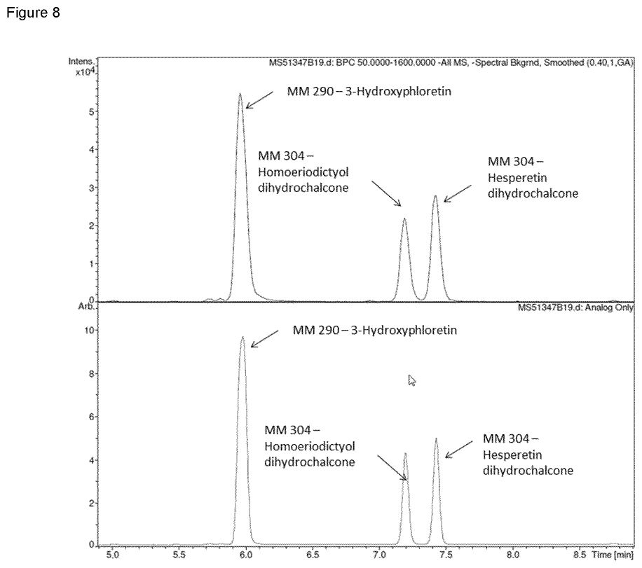

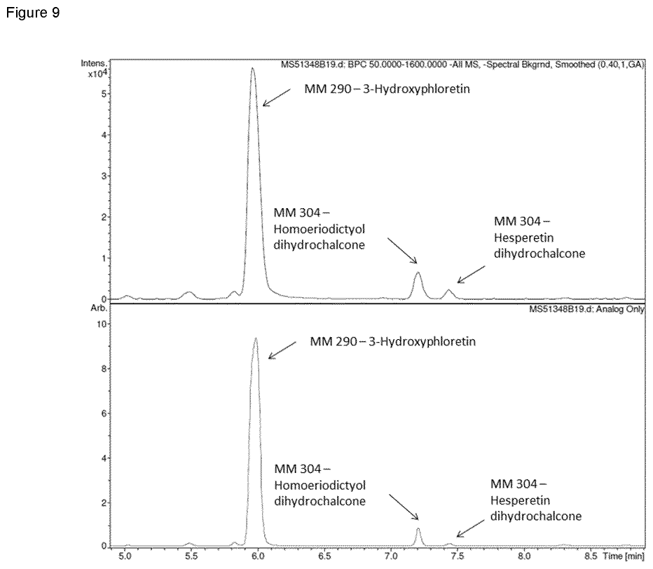

Cells from PPS-9010_SAM_MxSafC or PPS-9010_SAM_PsOMT were used to inoculate 10 ml of BMGYM medium. After incubation overnight (30° C., 200 rpm) 5 ml culture were centrifuged, the pellet was resuspended in 1.4 ml Tris-HCl buffer pH 7.5 and the cells were disintegrated with glass beads (0.25-0.5 mm diameter) in a vortexer. The lysate was then centrifuged and the supernatant was mixed with 3 mM 3-hydroxyphloretine, 3 mM S-adenosylmethionine, 0.67 mM MgCl2. The reaction mixture was incubated at 25° C. for 24 hours. After stopping the assay with 20% trichloroacetic acid (5.7% final concentration) the sample was centrifuged and the supernatant used for LC-MS analysis. The results of the biocatalysis are depicted in FIGS. 8 and 9.

### Example 8: Polishing of the Mixture

500 mL biocatalysis solution from Example 3 or Example 7 were extracted 1:1 (v/v) with ethyl acetate in the separating funnel. Subsequently, the organic phase was concentrated to dryness at the rotary evaporator (30° C., 100 mBar). The obtained extract was separated by flash chromatography at a Sepacore X10 plant (Büchi Germany). For this, approx. 200 mg of the extract was discharged onto a silica gel 60 (Merck, Germany) column and with a gradient of hexane (A)/ethyl acetate (B) (2% A-100% A in 120 min, at 20 mL/min). The fractions containing 3-hydroxylphloretine or a mixture of homoeriodictyol dihydrochalcone and hesperetin dihydrochalcone were then concentrated to dryness at the rotary evaporator (30° C., 100 mBar).

### Example 9: Sensory Analyses

As the biocatalytic methods established yielded promising amounts of both homoeriodictyol dihydrochalcone (HEDDC) and hesperetin dihydrochalcone (HC), the products, further purified and as directly obtained, were further subjected to a series of sensory analyses. To this end, HEDDC and HC were used in defined ratios (wt. %) starting from rather high 1,000:1 and 1:1,000, respectively down to a 50/50 mixture 1:1.

35 Various ratios were tested. It was found that the resulting final product strongly influenced the optimum weight ratio. We already found surprising results for a high HEDDC:HC in certain settings.

To standardize the protocols, a 1:1 ratio (10 ppm to 10 ppm) was used in a first comparative data set. This maximum dose will, however, not be necessary in all settings.

First, an aqueous solution with different weight ratios of HEDDC and HC in comparison to HC alone, homoeriodictyol alone (H) or HEDDC alone was tested. In all standardized taste settings, the flavour was categorized as stronger in vanilla. In certain settings, the testers also confirmed that the mixtures (when going near to the 1:1 ratio) was sweeter.

Secondly, sugars and acids were added to pinpoint the relevant effects. In one setting, 5 wt. of a sugar and 0.15% of an organic acid, usually citric acid, was tested. It was consistently confirmed that the HEDDC/HC mixture was always superior to HC alone in case at least an acid was added, as the sensory profile was categorized as much less astringent and sour. Depending on the ratios, also a richer head was affirmed.

Finally, we tested the addition of further sweetness enhancers, aroma agents and flavouring substances. With RebA, for example, and using a nearly equal HEDDC/HC ratio, a fuller flavour and a richer head was affirmed by the initial test in comparison to the same compositions using HC alone. Notably, we also tested whether the above effects could be adjusted by using higher amounts of HC alone. In all different test series, the combined HEDDC/HC mixture, however, always had a somehow synergistic effect which we could not mimic by adding more of HC or H alone.

1. A method for the biocatalytical manufacturing of homoeriodictyol dihydrochalcone and/or hesperetin dihydrochalcone, comprising the steps:
i) providing at least one first biocatalyst system comprising at least one oxidase or a sequence encoding the same, and at least one reductase or a sequence encoding the same,
ii) contacting the at least one first biocatalyst system with phloretin and/or its glycosides and incubating the mixture,
iii) obtaining 3-hydroxyphloretin,
iv) providing at least one second biocatalyst and optionally at least one methyl group donor,
v) contacting the at least one second biocatalyst provided in step iv) with the 3-hydroxyphloretin obtained in step iii) and optionally with the at least one methyl group donor provided in step iv) and incubating the mixture, and
vi) obtaining homoeriodictyol dihydrochalcone and/or hesperetin dihydrochalcone,
wherein the homoeriodictyol dihydrochalcone (1) and the hesperetin dihydrochalcone (2) have the following formula:

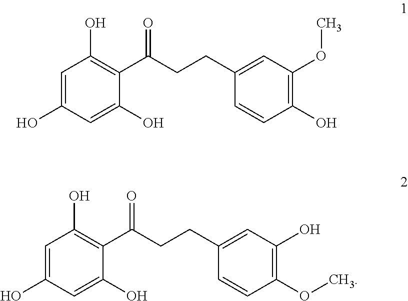 [SMILES: COc1cc(CCC(=O)c2c(O)cc(O)cc2O)ccc1O ; COc1ccc(CCC(=O)c2c(O)cc(O)cc2O)cc1O]

2. The method of claim 1, wherein the first and second of the at least one biocatalyst or biocatalyst system is or are provided as/in at least one of an enzyme, a purified enzyme, a cell lysate, a whole cell reaction or as a sequence encoding the biocatalyst, or a combination thereof.

3. The method of claim 1, wherein the at least one second biocatalyst is an O-methyltransferase or a sequence encoding the same.

4. The method of claim 1, wherein the at least one first and/or second biocatalyst system or biocatalyst is/are a purified or partially purified biocatalyst or biocatalyst system.

5. The method of claim 1, wherein the at least one first biocatalyst system comprises at least two sequences encoded by an amino acid sequence of SEQ ID NO: 8 and SEQ ID NO:10 or a nucleic acid sequence encoding the respective amino acid sequence, or a homologue thereof, or by an amino acid sequence having at least 90%, 91%, 92%, 93%, 94%, 95%, 96%, 97%, 98% or 99% sequence homology to an amino acid sequence according to SEQ ID NO: 8 and SEQ ID NO:10 or a nucleic acid sequence encoding the respective amino acid sequence, and wherein the at least one second biocatalyst is encoded by an amino acid sequence of SEQ ID NO: 14 or 16 or a homologue thereof, or a nucleic acid sequence encoding the respective amino acid sequence, or by an amino acid sequence having at least 90%, 91%, 92%, 93%, 94%, 95%, 96%, 97%, 98% or 99% sequence homology to an amino acid sequence of SEQ ID NO: 14 or SEQ ID NO: 16 or a nucleic acid sequence encoding the respective amino acid sequence.

6. The method of claim 1, wherein the at least one first biocatalyst system additionally comprises a Glucose-6-phosphate dehydrogenase (G6P) or a sequence encoding the same, wherein the at least one G6P is encoded by an amino acid sequence selected from the group consisting of SEQ ID NO: 46 and SEQ ID NO: 48, or a homologue thereof, or a nucleic acid sequence encoding the respective amino acid sequence, or by an amino acid sequence having at least 90%, 91%, 92%, 93%, 94%, 95%, 96%, 97%, 98% or 99% sequence homology to an amino acid sequence according to any one of SEQ ID NO: 46 and SEQ ID NO: 48 or a nucleic acid sequence encoding the respective amino acid sequence.

7. The method of claim 1, wherein the at least one oxidase of the first biocatalyst system is a CYP450 oxidase, and wherein the at least one reductase of the first biocatalyst system is a CYP450 reductase.

8. The method of claim 1, wherein the biocatalyst is produced by or present in a cell selected from the group consisting of Escherichia coli spp., Bacillus spp., Saccharomyces spp., Hansenula or Komagataella spp., Yarrowia spp., or Kluyveromyces spp.

9. The method of claim 1, wherein steps i) and ii), or steps i), ii), iv) and v), or steps iv) and v) are conducted simultaneously.

10. The method of claim 1, wherein the phloretin and/or its glycosides provided in step ii) and/or the 3-hydroxyphloretin obtained in step iii) is/are additionally purified or partially purified.

11. The method of claim 1, wherein the method comprises adding at least one methyl group donor, and wherein the at least one methyl group donor is selected from the combination of S-adenosylmethionin and/or methionine and a S-adenosylmethionine synthase (SAM), wherein the S-adenosylmethionine synthase has an amino acid sequence selected from the group consisting of SEQ ID NO: 12, SEQ ID NO: 38, SEQ ID NO: 40, SEQ ID NO: 42, SEQ ID NO: 44, or a homologue thereof, a nucleic acid sequence encoding the respective amino acid sequence or by an amino acid sequence having at least 90%, 91%, 92%, 93%, 94%, 95%, 96%, 97%, 98% or 99% sequence homology to an amino acid sequence according to any one of SEQ ID NO: 12, SEQ ID NO: 38, SEQ ID NO: 40, SEQ ID NO: 42, SEQ ID NO: 44 or a nucleic acid sequence encoding the respective amino acid sequence.

12. The method of claim 1, wherein the method is a method for the biocatalytical manufacturing of a mixture of homoeriodictyol dihydrochalcone and hesperetin dihydrochalcone, wherein step v) comprises obtaining a mixture of homoeriodictyol dihydrochalcone and hesperetin dihydrochalcone, and/or wherein the method comprises an additional step of purifying or partially purifying the obtained homoeriodictyol dihydrochalcone and/or hesperetin dihydrochalcone.

13. An O-methyltransferase, wherein the O-methyltransferase comprises at least one mutation in comparison to the sequence according to SEQ ID NO: 14, and wherein the O-methyltransferase is selected from the group consisting of SEQ ID NOs: 69 to 76, or a functional fragment thereof, or a sequence having at least 90%, 91%, 92%, 93%, 94%, 95%, 96%, 97%, 98%, or 99% sequence identity to the respective sequence of SEQ ID NOs: 69 to 76, or the functional fragment thereof, or a nucleic acid sequence encoding the O-methyltransferase or the functional fragment thereof.

14. A composition comprising
(a) a mixture of homoeriodictyol dihydrochalcone and hesperetin dihydrochalcone in a weight ratio of about 1,000:1 to 1:1,000, in a weight ratio of about 100:1 to 1:100, in a weight ratio of about 50:1 to 1:50, in a weight ratio of about 10:1 to 1:10, in a weight ratio of about 5:1 to 1:5, or in a weight ratio of about 1:1; and
(b) at least one of an acid, a further flavour agent, a sweetening agent, and/or water.

15. (canceled)

16. The method of claim 8, wherein the Escherichia coli spp. comprises E. coli BL21, E. coli MG1655, or E. coli W3110.

17. The method of claim 8, wherein the Bacillus spp. comprises Bacillus licheniformis, Bacillus subitilis, or Bacillus amyloliquefaciens.

18. The method of claim 8, wherein the Saccharomyces spp. comprises S. cerevisiae.

19. The method of claim 8, wherein the Hansenula or Komagataella spp. comprises K. phaffii or H. polymorpha.

20. The method of claim 8, wherein the Yarrowia spp. comprises Y. lipolytica.

21. The method of claim 8, wherein the Kluyveromyces spp, comprises K. lactis.
